(19) United States
(12) Patent Application Publication
(10) Pub. No.: US 2021/0010028 A1
(43) Pub. Date: Jan. 14, 2021

(54) INSECT CELL MANUFACTURED PARTIAL SELF-COMPLEMENTARY AAV GENOMES

(71) Applicant: VOYAGER THERAPEUTICS, INC., Cambridge, MA (US)

(72) Inventors: Eric D. Horowitz, Norwell, MA (US); Sylvain Cecchini, Westborough, MA (US); Robert Steininger, Cambridge, MA (US); David Dismuke, Cary, NC (US); Christopher J. Morrison, Arlington, MA (US)

(21) Appl. No.: 16/978,288

(22) PCT Filed: Mar. 6, 2019
(86) PCT No.: PCT/US2019/020892

§ 371 Date: Sep. 4, 2020

Related U.S. Application Data
(60) Provisional application No. 62/639,437, filed on Mar. 6, 2018.

(51) Int. Cl. C12N 15/86; C12N 9/88

(52) U.S. Cl. CPC C12N 15/86; C12N 9/88; C12N 2750/14152; C12N 2750/14143; C12Y 401/01028

(57) ABSTRACT

The present disclosure is directed to parvovirus genomes; plasmid vectors encoding parvovirus genomes, and particles and populations thereof; as well as methods of their production and use.

### CROSS REFERENCE TO RELATED APPLICATIONS

This application claims the benefit of U.S. Provisional Patent Application No. 62/639,437, filed Mar. 6, 2018, entitled INSECT CELL MANUFACTURED PARTIAL SELF-COMPLEMENTARY AAV GENOMES, the contents of which is incorporated herein by reference in its entirety.

### FIELD OF THE DISCLOSURE

The present invention relates generally to partial self-complementary parvovirus genomes, such as AAV genomes, and more specifically to partial self-complementary parvovirus genomes produced using insect cells, including parvovirus particles containing such genomes, methods of using such particles and methods of making and enriching for such particles.

### BACKGROUND

The single-stranded nature of the parvoviral genome, requires the use of cellular mechanisms to provide a complementary strand for gene expression. Recruitment of cellular factors and second strand synthesis are considered to be an important rate-limiting factor in the efficiency of transduction and gene expression in parvovirus particles. This problem can be circumvented by packaging both strands as a single duplex DNA molecule in the form of a self-complementary parvovirus genome. However, because of the packaging size limit of parvovirus particles (e.g., adeno-associated virus (AAV) has a packaging size limit of approximately 4.8 kb of a single stranded DNA), the size of the heterologous sequence that can be introduced into the genome for use as a therapeutic is further limited (e.g., self-complementary AAV has a limit of approximately 2.3 kb for the heterologous sequence). Thus, parvovirus therapeutic strategies that want to take advantage of the self-complementary parvovirus genome are limited to treating diseases where the therapeutic construct (e.g., protein needing to be expressed and the promoter driving its expression) has a size of that fits into approximately half of the already limited genome size of a parvovirus.

Thus, there exists a need for identifying and generating parvovirus particles that have larger capacities for therapeutic constructs while still not being subject to cellular rate limiting factors. This disclosure satisfies this need and provides related advantages.

### SUMMARY

Provided herein is a population of parvovirus (e.g., AAV) genomes comprising a high molecular weight parvovirus genome and a low molecular weight parvovirus genome, as well as plasmid vectors encoding the parvovirus genomes. In some embodiments, the low molecular weight parvovirus (e.g., AAV) genome includes a payload construct and parvovirus inverted terminal repeats (ITRs) flanking the payload construct, and the high molecular weight parvovirus (e.g., AAV) genome includes the payload construct and the parvovirus (e.g., AAV) ITRs and further contains an additional region flanking one of the ITRs, wherein the length of the region is less the entire length of the payload construct of the low molecular weight parvovirus genome. In particular embodiments, such a population of high molecular weight and low molecular weight parvovirus (e.g., AAV) genomes is produced by insect cells by, for example, using an Sf9/baculovirus insect cell system. In still further embodiments, provided herein is such a population of parvovirus (e.g., AAV) genomes wherein the population is enriched for high molecular weight parvovirus genomes. In yet another embodiment, provided herein is such a population of parvovirus (e.g., AAV) genomes wherein the population is enriched for low molecular weight parvovirus genomes.

Also provided herein is a partial self-complementary parvovirus (e.g., AAV) genome, as well as plasmid. vectors encoding the parvovirus genomes. In some embodiments, the partial self-complementary parvovirus (e.g., AAV) genome includes a payload construct, parvovirus (e.g., AAV) ITRs flanking the payload construct, and a self-complementary region flanking one of the ITRs, wherein the self-complementary region includes a nucleotide sequence that is complementary to the payload construct and a length that is less the entire length of the payload construct.

Further provided herein is a parvovirus particle having the partial self-complementary parvovirus (e.g., AAV) genome, as well as plasmid vectors encoding the parvovirus genomes, wherein the partial self-complementary parvovirus genome includes a payload construct, parvovirus ITRs flanking the payload construct, and a self-complementary region flanking one of the ITRs, wherein the self-complementary region includes a nucleotide sequence that is complementary to the payload construct and a length that is less the entire length of the payload construct.

Also provided herein is a population of parvovirus AAV) particles having at least two sub-populations (a first sub-population of parvovirus particles and a second sub-population of parvovirus particles), wherein the first sub-population of parvovirus particles each include the high molecular weight parvovirus (e.g., AAV) genome that can include a partial self-complementary parvovirus (e.g., AAV) genome described herein, and wherein the second sub-population of parvovirus (e.g , AAV) particles each include low molecular weight parvovirus (e.g., AAV) genome that can include a genome that does not include the nucleotide sequence that is complementary to a portion of the payload construct. In one embodiment, the population of parvovirus AAV) particles is enriched for parvovirus particles having a high molecular weight parvovirus (e.g., AAV) genome that can include a partial self-complementary parvovirus genome described herein.

Still further, provided herein is a population of parvovirus (e.g., AAV) particles produced by insect cells wherein the population is enriched for parvovirus particles each having high molecular weight parvovirus (e.g., AAV) genome that can include a self-complementary parvovirus genome described herein. Also provided herein is a population of parvovirus (e.g., AAV) particles produced by insect cells wherein the population is enriched for parvovirus particles each having a low molecular weight parvovirus (e.g., AAV) genome that can include a genome that does not include the nucleotide sequence that is complementary to a portion of the payload construct described herein.

Provided herein is a pharmaceutical composition including the parvovirus (e.g., AAV) particle having a high molecular weight parvovirus (e.g., AAV) genome that can have a partial self-complementary parvovirus genome described herein and a pharmaceutically acceptable carrier. Also provided herein is a pharmaceutical composition including a population of parvovirus (e.g., AAV) particles produced by insect cells wherein the population is enriched for parvovirus particles having a high molecular weight parvovirus (e.g., AAV) genome that can have a self-complementary parvovirus genome described herein. Still further provided herein is a pharmaceutical composition including a population of parvovirus (e.g., AAV) particles each having a high molecular weight parvovirus (e.g., AAV) genome that can include a partial self-complementary parvovirus genome described herein and a pharmaceutically acceptable carrier.

Still further provided is an insect cell having a high molecular weight parvovirus (e.g., AAV) genome that can include a partial self-complementary parvovirus (e.g., AAV) genome described herein.

Provided herein is a method of making a population of parvovirus (e.g., AAV) particles that can include: (a) culturing insect cells with plasmid vectors encoding the parvovirus genomes of the present disclosure; (b) culturing insect cells with the parvovirus genomes to produce a population of parvovirus particles described herein; and (c) harvesting the population of parvovirus particles produced by the insect cells, wherein the harvested population of parvovirus particles include parvovirus particles having; the high molecular weight parvovirus genome that can include a partial self-complementary parvovirus genome described herein. In some embodiments, the population of parvovirus (e.g., AAV) particles produced by the method is enriched for the parvovirus particles that have the high molecular weight parvovirus genome that can include a partial self-complementary parvovirus genome described herein.

### BRIEF DESCRIPTION OF THE FIGURES

FIG. 1 illustrates representative, non-limiting vector genomes in accordance some embodiments described herein.

FIG. 2 illustrates a schematic representation of an example vector, and an image of lanes from a denaturing gel of AAV2 vectors.

FIG. 3A illustrates an example plot of refractive indices of AAV2 viral fractions.

FIG. 3B illustrates a plot of qPCR titers of vector fractions.

FIG. 4A shows an image of a denaturing gel of AAV2 vector fractions.

FIG. 4B shows the relative amounts of high molecular weight (High MW) and low molecular weight (Low MW) forms.

FIG. 5A shows an image of a gel separating digested vector DNAs. A description each identified band in the image (a) to (p) can be found in Table 1.

FIG. 5B shows a schematic depiction of the predicted transgene-containing vector genome structures and cleavage sites.

FIG. 6A show an image of a gel comparing PCR amplicons from high molecular weight (High MW PCR) and low molecular weight (Low MW PCR) forms.

FIG. 6B illustrates an example schematic of the hAADC transgene in a viral vector genome that as sequenced.

FIG. 7 illustrates a plot of vector genome titer measurements by qPCR.

FIG. 8 shows an example image of a Western blot for expression of an AADC transgene.

FIG. 9 illustrates a plot of vector titer measurements for fractions of an example population of AAV2 vectors (top), and an image of a denaturing gel for selected fractions (bottom).

FIG. 10 shows an example image of a Western blot for expression of an AADC transgene.

### DETAILED DESCRIPTION

### I. OVERVIEW

The compositions and methods provided herein are based, at least in part, on the identification and characterization of a partial self-complementary parvovirus genome in the context of AAV produced by an insect cell (e.g., Sf9/baculovirus) system. Accordingly, the present disclosure provides compositions and methods for the production of parvovirus (e.g., AAV) particles having a genome that includes a heterologous sequence for gene expression (e.g., a payload construct) that is greater than 2.3 kb in length (the size limit for a full length self-complementary parvovirus (e.g., AAV) genome) with improved gene expression. Thus, in some aspects, the compositions and methods provided herein allow for use of a larger heterologous sequence in the parvovirus (e.g., AAV) genome as compared to a full length self-complementary parvovirus (e.g., AAV) genome, while still allowing for higher gene expression levels as compared to particles having only a traditional fully single stranded DNA genome.

The phrase “baculovirus expression vector” or “BEV” refers to a baculovirus plasmid or bacmid having a viral construct for expression of non-structural and structural proteins or a payload construct described herein. Methods for introducing such constructs into a baculovirus plasmid or bacmid are well known in the art, which can include use of a transposon donor/acceptor system. A “baculovirus infected insect cell” or “BBC” refers to an insect cell that has been infected with a BEV.

When hybridization occurs in an antiparallel configuration between two single-stranded polynucleotides, the reaction is called annealing and those polynucleotides are described as “complementary.” A polynucleotide can be “complementary” to another polynucleotide, if hybridization can occur between the first polynucleotide and the second polynucleotide. “Complementarity” (the degree that one polynucleotide is complementary with another) is quantifiable in terms of the proportion of bases in opposing strands that are expected to form hydrogen bonding with each other, according to generally accepted base-pairing rules.

As used herein, the term “enriched” and any grammatical equivalent thereof means to improve the quality of a composition or population. As a non-limiting example, a sample can be enriched by increasing the proportion of a particular agent in the sample. In the context of the parvovirus particles and populations described herein and parvovirus genomes and populations described herein, the amount of desired particles or genomes in a given population of parvovirus particles or genomes can be enriched for as compared to a population of parvovirus particles or genomes produced using a different system (e.g., HEK293 triple transfection production system). Alternatively or additionally, the amount of desired particles or genomes in a given population of parvovirus particles or genomes can be enriched for as compared to the same population of parvovirus particles or genomes prior to its being enriched.

As used herein, the term “flanking” as used in the context of the features, regions and/or sequences including a parvovirus genome described herein means that the feature, region and/or sequence is contiguously situated on each side of or on one side of another feature, region and/or sequence.

As used herein, the phrase “high molecular weight parvovirus genome” means a parvovirus (e.g., AAV) genome that, when assayed, has more nucleotides than expected for a single stranded parvovirus genome. A high molecular weight parvovirus genome can, for example, have a molecular weight equivalent to more than a monomer parvovirus genome, but less than two monomer parvovirus genomes. Methods that can be used to assay for the presence of a high molecule weight parvovirus genome include, but are not limited to, denaturing (e.g., alkaline) gel electrophoresis and Southern blotting.

The term “insect cell” used herein means any insect cell that allows for replication of parvovirus and which can be maintained in culture and infected with baculovirus expression vector in accordance with the present disclosure and standard techniques. Non-limiting examples of insect cell lines include Spodoptera frugiperda pupal ovarian cell lines (e.g., Sf9 or Sf21), drosophila cell lines, or mosquito cell lines, such as Aedes albopictus derived cell lines.

As used herein, the phrase “inverted terminal repeat” or “ITR” means the polynucleotide sequence found at the ends of parvovirus genomes that form a hairpin, which contributes to the genome's ability to self-prime (allowing for primase-independent synthesis of the complementary second DNA strand) and provides for encapsidation of the genome into a parvovirus particle. An ITR can be a wild-type ITR, which can be 145 bases in length, or a variant thereof, for example, a 142 nucleotide variant thereof.

As used herein, the term “isolated” or “purified” when used in reference to a compound, substance or entity (e.g., a genome, particle, cell or population thereof) means that it is separated from other components and carries with it the understanding that the separation was carried out by the hand of man. An isolated compound, substance or entity can be one that has been separated from at least one of the components with which it was previously associated (whether in nature or in a prior composition). Isolated compounds, substances or entities can have varying levels of purity in reference to the components from which they have been associated. Isolated compounds, substances or entities can be separated from at least about 10%, about 20%, about 30%, about 40%, about 50%, about 60%, about 70%, about 80%, about 90%, or more of the components with which they were initially associated. An isolated compound, substance or entity can be more than about 80%, about 85%, about 90%, about 91%, about 92%, about 93%, about 94%, about 95%, about 96%, about 97%, about 98%, about 99%, or more than about 99% pure. As used herein, a compound, substance or entity is “pure” if it is detectably free of other components or only includes trace amounts of the other components from which it was separated from. A “substantially isolated” compound, substance or entity (e.g., a genome, particle, cell or population thereof) contains at least about 50%, at least about 60%, at least about 70%, at least about 80%, at least about 90%, at least about 95%, at least about 97%, or at least about 99% by weight of the compound, substance or entity of interest.

As used herein, the phrase “low molecular weight parvovirus genome” means a parvovirus (e.g., AAV) genome that, when assayed, has the number of nucleotides than are expected for a single stranded parvovirus genome. A low molecular weight parvovirus genome can, for example, have a molecular weight equivalent to a monomer parvovirus genome. Methods that can be used to assay for the presence of a low molecule weight parvovirus genome include, but are not limited to, denaturing (e.g., alkaline) gel electrophoresis and Southern blotting.

The phrase “modulatory nucleic acid” refers to an RNA sequence produced by a payload construct that modulates (e.g., increases or decreases) the expression of a protein or activity of a molecule in a cell. A modulatory nucleic acid can function through the process of RNA interference (RNAi), which inhibits gene expression or translation by neutralizing mRNA molecules. Non-limiting examples of modulatory nucleic acids include tRNA, rRNA, tmRNA, miRNA, siRNA, piRNA, shRNA, antisense RNA, double stranded RNA, snRNA, snoRNA, and/or long non-coding RNA (lncRNA).

The phrase “non-structural parvovirus proteins” means the proteins that are required for parvovirus replication, including site specific endonuclease and helicase activity, DNA replication and activation of promoters during transcription, or proteins that are required for assembly of the capsid of a parvovirus particle. In the context of AAV, the rep gene encodes the non-structural Rep proteins of Rep78, Rep68, Rep52 and Rep40, and the ORF2 of the cap gene encodes the non-structural Assembly-Activating Protein (AAP).

The term “parvovirus” as used herein refers to DNA animal viruses that contain a linear, single-stranded DNA genome and encompasses the family Parvoviridae, including autonomously-replicating parvoviruses and dependoviruses. The autonomous parvoviruses include members of the genera Parvovirus, Erythrovirus, Densovirus, Iteravirus, and Contravirus. Exemplary autonomous parvoviruses include, but are not limited to, mouse minute virus, bovine parvovirus, canine parvovirus, chicken parvovirus, panleukopenia virus, feline parvovirus, goose parvovirus, and B19 virus. Other autonomous parvoviruses are known to those skilled in the art. The genus Dependovirus contains the adeno-associated viruses (AAV) including, but not limited to, the following serotypes AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV-130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAV-DJ, AAV-DJ8, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10 and/or Japanese AAV 10 serotypes, and variants thereof.

As used herein, a “parvovirus genome” or “recombinant parvovirus genome” is a parvovirus (e.g., AAV) genome having at least two ITRs, which can have a nucleotide sequence (e.g., payload construct), heterologous or foreign to the native parvovirus genome, inserted into it.

As used herein, a “particle” in the context of a virus, for example a parvovirus (e.g., AAV), is a virus that includes at least two components, a protein capsid component and a polynucleotide sequence (e.g., genome enclosed within the capsid component). A “recombinant parvovirus particle” includes a recombinant parvovirus genome packaged within parvovirus capsid.

As used herein, “payload construct” is one or more nucleotide sequences encoding or including a payload molecule of interest (e.g., a transgene, a polynucleotide encoding a protein or a modulatory nucleic acid) that, in the context of a parvovirus genome, is flanked on one or both sides by an ITR.

As used herein, the term “pharmaceutically acceptable carrier” includes any of the standard pharmaceutical carriers, such as a phosphate buffered saline solution, water, and emulsions, such as an oil/water or water/oil emulsion, and various types of wetting agents. The compositions also can include excipients, stabilizers, adjuvants and preservatives. For examples of carriers, excipients, stabilizers and adjuvants, see Remington: The Science and Practice of Pharmacy, 22nd Revised Ed., Pharmaceutical Press, 2012.

As used herein, the terms “protein of interest” or “desired protein” or the like means a protein provided herein and fragments, mutants, variants, and alterations thereof.

The phrase “relative molar amount” when used in the context of parvovius particles or genomes in a given population refers to the relative relationship of a population of parvovirus particles or genomes to a reference population of parvovirus particles or genomes based on the calculated molar concentration (molarity) of the reference population. Methods of determining the concentration of a population of parvovirus particles or genomes in a sample are well known in the art, including, without limitation, native agarose gel electrophoresis, denaturing agarose gel electrophoresis, capillary electrophoresis, capillary gel electrophoresis, ion exchange chromatography, size exclusion chromatography, ultracentrifugation and analytical ultracentrifugation, which can include titration of a sample.

As used herein, a “self-complementary parvovirus genome” is a single stranded polynucleotide having, in the 5′ to 3′ direction, a first parvovirus ITR sequence, a heterologous sequence (e.g., payload construct), a second parvovirus ITR sequence, a second heterologous sequence, wherein the second heterologous sequence is complementary to the first heterologous sequence, and a third parvovirus ITR sequence. In contrast to a self-complementary genome, a “partial self-complementary genome” does not include three parvovirus ITRs and the second heterologous sequence that is complementary to the first heterologous sequence has a length that is less than the entire length of the first heterologous sequence (e.g., payload construct). Accordingly, a partial self-complementary genome is a single stranded polynucleotide having, in the 5′ to 3′ direction or the 3′ to 5′ direction, a first parvovirus ITR sequence, a heterologous sequence (e.g., payload construct), a second parvovirus ITR sequence, and a self-complementary region that is complementary to a portion of the heterologous sequence and has a length that is less than the entire length the heterologous sequence.

The phase “structural parvovirus proteins” means the proteins that form the components of a capsid of a parvovirus particle. In the context of AAV, the cap gene encodes three structural proteins, VP1, VP2 and VP 3.

### II. PARVOVIRUS GENOMES AND PARTICLES

The present disclosure provides a population of parvovirus (e.g., AAV) genomes, as well as plasmid vectors encoding the parvovirus genomes, having a high molecular weight parvovirus genome and a low molecular weight parvovirus genome. In some aspects, the low molecular weight parvovirus (e.g., AAV) genome includes a payload construct and parvovirus ITRs flanking the payload construct. In some aspects, the high molecular weight parvovirus (e.g., AAV) genome includes the payload construct and the parvovirus (e.g., AAV) ITRs and further includes an additional region flanking one of the ITRs. In some aspects, the length of the region flanking one of the ITRs is less than the entire length of the payload construct of the low molecular weight parvovirus genome. In a particular aspect, such a population of high molecular weight and low molecular weight parvovirus (e.g., AAV) genomes is produced by insect cells (e.g., Sf9), such as by use of an Sf9/baculovirus insect cell system.

In still further embodiments, provided herein is a population of parvovirus (e.g., AAV) genomes wherein the population is enriched for high molecular weight parvovirus genomes. In yet further embodiments, provided herein is a population of parvovirus (e.g., AAV) genomes wherein the population is enriched for low molecular weight parvovirus genomes.

The present disclosure also provides a population of parvovirus (e.g., AAV) particles having a population of particles having a high molecular weight parvovirus genome and a population of particles having a low molecular weight parvovirus genome. In some aspects, the population of parvovirus particles having low molecular weight parvovirus (e.g., AAV) genomes include a payload construct and parvovirus ITRs flanking the payload construct. In some aspects, the population of parvovirus particles having high molecular weight parvovirus (e.g., AAV) genomes includes the payload construct and the parvovirus (e.g., AAV) ITRs and further includes an additional region flanking one of the ITRs. In some aspects, the length of the region flanking one of the ITRs in the population of parvovirus particles is less than the entire length of the payload construct of the low molecular weight parvovirus genome. In a particular aspect, such a population of parvovirus particles having high molecular weight and low molecular weight parvovirus (e.g., AAV) genomes is produced by insect cells (e.g., Sf9), such as by use of an Sf9/baculovirus insect cell system.

In still further embodiments, provided herein is a population of parvovirus particles having parvovirus (e.g., AAV) genomes wherein the population is enriched for parvovirus particles having high molecular weight parvovirus genomes. In yet further embodiments, provided herein is a population of parvovirus particles having parvovirus (e.g., AAV) genomes wherein the population is enriched for parvovirus particles having low molecular weight parvovirus genomes.

The present disclosure also provides partial self-complementary parvovirus (e.g., AAV) genomes, plasmid vectors encoding the parvovirus genomes, and parvovirus (e.g., AAV) particles including such genomes.

In some embodiments, provided herein is a partial self-complementary parvovirus genome as described herein. In some embodiments, provided herein is a plasmid vector which includes a nucleotide sequence encoding a parvovirus genome of the present disclosure, Accordingly, in some aspects, provided herein is a partial self-complementary parvovirus genome including a payload construct, parvovirus ITRs flanking the payload construct, and a self-complementary region flanking one of the ITRs. In some aspects, the self-complementary region includes a nucleotide sequence that is complementary to the payload construct. In some aspects, the self-complementary region has a length that is less the entire length of the payload construct.

In some embodiments, the parvovirus genome provided herein is an AAV genome. In a further embodiment, the AAV genome is any one of the well-known serotypes of AAV in the art, such as AAV2.

In some embodiments, the parvovirus genome provided herein includes payload construct encodes a protein of interest or produces a modulatory nucleic acid has described herein.

In some embodiments, the parvovirus genome provided herein has a minimum size of the payload construct. Accordingly, in some aspects, the payload construct is 2.3 kb or more in length. In some aspects, the payload construct is 2.4 kb or more in length. In some aspects, the payload construct is 2.5 kb or more in length. In some aspects, the payload construct is 2.6 kb or more in length. In some aspects, the payload construct is 2.7 kb or more in length. In some aspects, the payload construct is 2.8 kb or more in length. In some aspects, the payload construct is 2.9 kb or more in length. In some aspects, the payload construct is 3.0 kb or more in length. In some aspects, the payload construct is 3.1 kb or more in length. In some aspects, the payload construct is 3.2 kb or more in length. In some aspects, the payload construct is 3.3 kb or more in length. In some aspects, the payload construct is 3.4 kb or more in length. In some aspects, the payload construct is 3.5 kb or more in length.

In some embodiments, the self-complementary region of the parvovirus genome provided herein has a minimum length, while still having a length that is less the entire length of the payload construct. Accordingly, in some aspects, the self-complementary region is at least 50 bases in length. In some aspects, the self-complementary region is at least 100 bases in length. In some aspects, the self-complementary region is at least 200 in length. In some aspects, the self-complementary region is at least 300 bases in length. In some aspects, the self-complementary region is at least 400 bases in length. In some aspects, the self-complementary region is at least 500 bases in length. In some aspects, the self-complementary region is at least 600 bases in length. In some aspects, the self-complementary region is at least 700 bases in length. In some aspects, the self-complementary region is at least 800 bases in length. In some aspects, the self-complementary region is at least 900 bases in length. In some aspects, the self-complementary region is at least 1,000 bases in length.

In some embodiments, because the self-complementary region of the parvovirus genome has a length that is less the entire length of the payload construct, the self-complementary region has a maximum length. Accordingly, in some aspects, the self-complementary region has a length of no more than 2.2 kb. In some aspects, the self-complementary region has a length of no more than 2.1 kb. In some aspects, the self-complementary region has a length of no more than 2.0 kb. In some aspects, the self-complementary region has a length of no more than 1.9 kb. In some aspects, the self-complementary region has a length of no more than 1.8 kb. In some aspects, the self-complementary region has a length of no more than 1.7 kb. In some aspects, the self-complementary region has a length of no more than 1.6 kb. In some aspects, the self-complementary region has a length of no more than 1.5 kb. In some aspects, the self-complementary region has a length of no more than 1.4 kb. In some aspects, the self-complementary region has a length of no more than 1.3 kb. In some aspects, the self-complementary region has a length of no more than 1.1 kb.

In some embodiments, the self-complementary region has a length that is sufficient to provide for higher activity of the encoded protein or modulatory nucleic acid of the payload construct as compared to a fully single stranded genome. Accordingly, in some aspects, the self-complementary region has a length between 50 bases and 2.0 kb. In some aspects, the self-complementary region has a length between 100 bases and 1.5 kb. In some aspects, the self-complementary region has a length between 1.0 kb and 2.0 kb.

In some embodiments, the partial self-complementary genome described herein has a total length (e.g., including the ITRs and payload construct) of no more than 4.5 kb, 4.6 kb, 4.7 kb or 4.8 kb.

In some embodiments, provided herein is a parvovirus particle having a partial self-complementary parvovirus genome as described herein. Accordingly, in some aspects, provided herein is a parvovirus particle having a partial self-complementary parvovirus genome including a payload construct, parvovirus ITRs flanking the payload construct, and a self-complementary region flanking one of the ITRs. In some aspects, the self-complementary region of the parvovirus particle includes a nucleotide sequence that is complementary to the payload construct. In some aspects, the self-complementary region of the parvovirus particle has a length that is less the entire length of the payload construct.

In some embodiments, the parvovirus particle provided herein is an AAV particle. In a further embodiment, the AAV particle is any one of the well-known serotypes of AAV in the art, such as AAV2.

In some embodiments, the parvovirus particle provided herein includes a payload construct that encodes a protein of interest or produces a modulatory nucleic acid has described herein.

In some embodiments, the parvovirus particle provided herein has a minimum size of the payload construct. Accordingly, in some aspects, the parvovirus particle has a payload construct of 2.3 kb or more in length. In some aspects, the parvovirus particle has a payload construct of 2.4 kb or more in length. In some aspects, the parvovirus particle has a payload construct of 2.5 kb or more in length. In some aspects, the parvovirus particle has a payload construct of 2.6 kb or more in length. In some aspects, the parvovirus particle has a payload construct of 2.7 kb or more in length. In some aspects, the parvovirus particle has a payload construct of 2.8 kb or more in length. In some aspects, the parvovirus particle has a payload construct of 2.9 kb or more in length. In some aspects, the parvovirus particle has a payload construct of 3.0 kb or more in length. In some aspects, the parvovirus particle has a payload construct of 3.1 kb or more in length. In some aspects, the parvovirus particle has a payload construct of 3.2 kb or more in length. In some aspects, the parvovirus particle has a payload construct of 3.3 kb or more in length. In some aspects, the parvovirus particle has a payload construct of 3.4 kb or more in length. In some aspects, the parvovirus particle has a payload construct of 3.5 kb or more in length.

In some embodiments, the self-complementary region of the parvovirus particle provided herein has a minim-um length, while still having a length that is less the entire length of the payload construct, Accordingly, in some aspects, the self-complementary region of the parvovirus particle is at least 50 bases in length. In some aspects, the self-complementary region of the parvovirus particle is at least 100 bases in length. In some aspects, the self-complementary region of the parvovirus particle is at least 200 in length. In some aspects, the self-complementary region of the parvovirus particle is at least 300 bases in length. In some aspects, the complementary of the parvovirus particle region is at least 400 bases in length. In some aspects, the self-complementary region of the parvovirus particle is at least 500 bases in length. In some aspects, the self-complementary region of the parvovirus particle is at least 600 bases in length. In some aspects, the self-complementary region of the parvovirus particle is at least 700 bases in length. In some aspects, the self-complementary region of the parvovirus particle is at least 800 bases in length. In some aspects, the self-complementary region of the parvovirus particle is at least 900 bases in length. In some aspects, the self-complementary region of the parvovirus particle is at least 1,000 bases in length.

In some embodiments, because the self-complementary region of the parvovirus particle has a length that is less the entire length of the payload construct, the self-complementary region of the parvovirus particle has a maximum length. Accordingly, in some aspects, the self-complementary region of the parvovirus particle has a length of no more than 2.2 kb. In some aspects, the self-complementary region of the parvovirus particle has a length of no more than 2.1 kb. In some aspects, the self-complementary region of the parvovirus particle has a length of no more than 2.0 kb. In some aspects, the self-complementary region of the parvovirus particle has a length of no more than 1.9 kb. In some aspects, the self-complementary region of the parvovirus particle has a length of no more than 1.8 kb. In some aspects, the self-complementary region of the parvovirus particle has a length of no more than 1.7 kb. In some aspects, the self-complementary region of the parvovirus particle has a length of no more than 1.6 kb, in some aspects, the self-complementary region has a length of no more than 1.5 kb. In some aspects, the self-complementary region of the parvovirus particle has a length of no more than 1.4 kb. In some aspects, the self-complementary region of the parvovirus particle has a length of no more than 1.3 kb. In some aspects, the self-complementary region of the parvovirus particle has a length of no more than 1.1 kb.

In some embodiments, the self-complementary region of the parvovirus particle has a length that is sufficient to provide for higher activity of the encoded protein or modulatory nucleic acid of the payload construct as compared to a fully single stranded genome. Accordingly, in some aspects, the self-complementary region of the parvovirus particle has a length between 50 bases and 2.0 kb. In some aspects, the self-complementary region of the parvovirus particle has a length between 100 bases and 1.5 kb. In some aspects, the self-complementary region of the parvovirus particle has a length between 1.0 kb and 2.0 kb.

In some embodiments, the partial self-complementary genome of the parvovirus particle described herein has a total length (e.g., including the ITRs and payload construct) of no more than 4.8 kb.

In some embodiments, provided herein is a population of parvovirus particles as described herein, Accordingly, in some aspects, provided herein is a population of parvovirus particles that includes a first sub-population of parvovirus particles each including a parvovirus genome described herein. In some aspects, the first sub-population of parvovirus particles is enriched with such parvovirus particles. In some aspect, the first sub-population of parvovirus particles is removed from the population.

In some embodiments, provided herein is a population of parvovirus particles that includes a first sub-population of parvovirus particles arid a second sub-population of parvovirus particles. In some aspects, the first sub-population of parvovirus particles each have a high molecular weight parvovirus (e.g., AAV) genome that can include a partial self-complementary parvovirus genome described herein and the second sub-population of parvovirus particles each include a low molecular weight parvovirus (e.g., AAV) genome that can include a genome that does not include the nucleotide sequence that is complementary to a portion of the payload construct as described herein. In a further aspect, the first sub-population of parvovirus particles is substantially isolated from the second sub-population of parvovirus particles. In still another aspect, the second sub-population of parvovirus particles is isolated from the first sub-population.

In some embodiments, the relative molar amount of the first sub-population of parvovirus particles is at least 10% of the second sub-population of parvovirus particles. In some aspects, the relative molar amount of the first sub-population of parvovirus particles is at least 20% of the second sub-population of parvovirus particles. In some aspects, the relative molar amount of the first sub-population of parvovirus particles is at least 30% of the second sub-population of parvovirus particles. In some aspects, the relative molar amount of the first sub-population of parvovirus particles is at least 40% of the second sub-population of parvovirus particles. In some aspects, the relative molar amount of the first sub-population of parvovirus particles is at least 50% of the second sub-population of parvovirus particles. In some aspects, the relative molar amount of the first sub-population of parvovirus particles is at least 60% of the second sub-population of parvovirus particles. In some aspect, the relative molar amount of the first sub-population is at least 70% of the second sub-population of parvovirus particles. In some aspects, the relative molar amount of the first sub-population of parvovirus particles is at least 80% of the second sub-population of parvovirus particles. In some aspects, the relative molar amount of the first sub-population of parvovirus particles is at least 90% of the second sub-population of parvovirus particles. In some aspects, the relative molar amount of the first sub-population of parvovirus particles is at least 100% of the second sub-population of parvovirus particles. In some aspects, the relative molar amount of the first sub-population of parvovirus particles is at least 110% of the second sub-population of parvovirus particles. In some aspects, the relative molar amount of the first sub-population of parvovirus particles is at least 150% of the second sub-population of parvovirus particles. In some aspects, the relative molar amount of the first sub-population of parvovirus particles is at least 200% of the second sub-population of parvovirus particles. In some aspects, the relative molar amount of the first sub-population of parvovirus particles is at least 300% of the second sub-population of parvovirus particles. In some aspects, the relative molar amount of the first sub-population of parvovirus particles is at least 400% of the second sub-population of parvovirus particles. In some aspects, the relative molar amount of the first sub-population of parvovirus particles is at least 500% of the second sub-population of parvovirus particles. In some aspects, the relative molar amount of the first sub-population of parvovirus particles is at least 600% of the second sub-population of parvovirus particles. In some aspects, the relative molar amount of the first sub-population of parvovirus particles is at least 700% of the second sub-population of parvovirus particles. In some aspects, the relative molar amount of the first sub-population of parvovirus particles is at least 800% of the second sub-population of parvovirus particles. In some aspects, the relative molar amount of the first sub-population of parvovirus particles is at least 900% of the second sub-population of parvovirus particles. In some aspects, the relative molar amount of the first sub-population of parvovirus particles is at least 1,000% of the second sub-population of parvovirus particles.

In some embodiments, an increase in the relative molar amount of the second sub-population of parvovirus particles is desirable. Accordingly, in some aspects, the relative molar amount of the second sub-population of parvovirus particles is at least 10% of the first sub-population of parvovirus particles. In some aspects, the relative molar amount of the second sub-population of parvovirus particles is at least 20% of the first sub-population of parvovirus particles. In some aspects, the relative molar amount of the second sub-population of parvovirus particles is at least 30% of the first sub-population of parvovirus particles. In some aspects, the relative molar amount of the second sub-population of parvovirus particles is at least 40% of the first sub-population of parvovirus particles. In some aspects, the relative molar amount of the second sub-population of parvovirus particles is at least 50% of the first sub-population of parvovirus particles. In some aspects, the relative molar amount of the second sub-population of parvovirus particles is at least 60% of the first sub-population of parvovirus particles. In some aspect, the relative molar amount of the second sub-population is at least 70% of the first sub-population of parvovirus particles. In some aspects, the relative molar amount of the second sub-population of parvovirus particles is at least 80% of the first sub-population of parvovirus particles. In some aspects, the relative molar amount of the second sub-population of parvovirus particles is at least 90% of the first sub-population of parvovirus particles. In some aspects, the relative molar amount of the second sub-population of parvovirus particles is at least 100% of the first sub-population of parvovirus particles. In some aspects, the relative molar amount of the second sub-population of parvovirus particles is at least 110% of the first sub-population of parvovirus particles. In some aspects, the relative molar amount of the second sub-population of parvovirus particles is at least 150% of the first sub-population of parvovirus particles. In some aspects, the relative molar amount of the second sub-population of parvovirus particles is at least 200% of the first sub-population of parvovirus particles, In some aspects, the relative molar amount of the second sub-population of parvovirus particles is at least 300% of the first sub-population of parvovirus particles. In some aspects, the relative molar amount of the second sub-population of parvovirus particles is at least 400% of the first sub-population of parvovirus particles. In some aspects, the relative molar amount of the second sub-population of parvovirus particles is at least 500% of the first sub-population of parvovirus particles. In some aspects, the relative molar amount of the second sub-population of parvovirus particles is at least 600% of the first sub-population of parvovirus particles. In some aspects, the relative molar amount of the second sub-population of parvovirus particles is at least 700% of the first sub-population of parvovirus particles. In some aspects, the relative molar amount of the second sub-population of parvovirus particles is at least 800% of the first sub-population of parvovirus particles. In some aspects, the relative molar amount of the second sub-population of parvovirus particles is at least 900% of the first sub-population of parvovirus particles. In some aspects, the relative molar amount of the second sub-population of parvovirus particles is at least 1,000% of the first sub-population of parvovirus particles.

### III. PAYLOADS

The payload construct of the present disclosure includes a nucleic acid sequence (e.g., transgene) encoding at least one payload molecule, such as a protein or a modulatory nucleic acid. The payload molecule can include any nucleic acid produced by the parvovirus genome that is produced in accordance with the present disclosure for expression in a target cell transduced or contacted with the parvovirus particle.

According to the present disclosure, the parvovirus genome can include a payload construct that encodes a payload molecule. The payload molecule can include a protein, an RNA molecule, or any other gene product that is desired for expression in the target cell. The payload construct can include a combination of coding and non-coding nucleic acid sequences.

In one embodiment, the payload construct includes more than one nucleic acid sequence encoding more than one payload molecule of interest. In such an embodiment, a payload construct encoding more than one payload molecule can be replicated and packaged into a parvovirus particle. A target cell transduced with a parvovirus particle including more than one payload construct can express each of the payload molecules in a single cell.

In some embodiments, the payload construct sequence can encode a coding or non-coding RNA.

Where the payload construct sequence encodes a polypeptide, the polypeptide can be a peptide or protein. A protein encoded by the payload construct sequence can include a secreted protein, an intracellular protein, an extracellular protein, and/or a membrane protein. The encoded proteins can be structural or functional. Proteins encoded by the payload construct or payload construct include, but are not limited to, mammalian proteins, for example, human proteins. The virus particles described herein containing payload constructs sequences can, for example, be used in the fields of human and animal disease applications and in a variety of in vivo and in vitro settings, for example in payload molecule production or manufacturing settings.

In some embodiments, the payload construct encodes a messenger RNA (mRNA). As used herein, the term “messenger RNA” (mRNA) refers to any polynucleotide which encodes a polypeptide of interest and which is capable of being translated to produce the encoded polypeptide of interest in vitro, in vivo, in situ or ex vivo.

Traditionally, the basic components of an mRNA molecule include at least a coding region, a 5′UTR, a 3′UTR, a 5′ cap and a poly-A tail. According to the present disclosure, payload constructs encoding mRNA can include a coding region only. They can also include a coding region and at least one UTR. They can also include a coding region, 3′UTR and a poly-A tail.

In one embodiment a polypeptide encoded by a payload construct is between 50-5000 amino acids in length. In some embodiments the protein encoded is between 50-2000 amino acids in length. In some embodiments the protein encoded is between 50-1500 amino acids in length. In some embodiments the protein encoded is between 50-1000 amino acids in length. In some embodiments the protein encoded is between 50-800 amino acids in length. In some embodiments the protein encoded is between 50-600 amino acids in length. In some embodiments the protein encoded is between 50-400 amino acids in length. In some embodiments the protein encoded is between 50-200 amino acids in length. In some embodiments the protein encoded is between 50-100 amino acids in length.

In some embodiments a peptide encoded by a payload construct is between 4-50 amino acids in length. In one embodiment, the peptide is a tetrapeptide, a pentapeptide, a hexapeptide, a heptapeptide, an octapeptide, a nonapeptide, or a decapeptide. In another embodiment, the peptide is a peptide of 2-30 amino acids, e.g. 5-30, 10-30, 2-25, 5-25, 10-25, or 10-20 amino acids. In yet another embodiment, the peptide is least 11, 12, 13, 14, 15, 17, 20, 25 or 30 amino acids, or the peptide is no longer than 50 amino acids, e.g. no longer than 35, 30, 25, 20, 17, 15, 14, 13, 12, 11 or 10 amino acids.

An RNA encoded by the payload construct can include an mRNA, tRNA, rRNA, tmRNA, miRNA, siRNA, piRNA, shRNA antisense RNA, double stranded RNA, snRNA, snoRNA, and long non-coding RNA (lncRNA). Examples of such lncRNA molecules and RNAi constructs designed to target such lncRNA any of which can be encoded in the payload constructs are taught in International Publication, WO2012/018881 A2, the contents of which are incorporated herein by reference in their entirety.

In one embodiment, the payload construct encodes a microRNA or miRNA as the payload molecule. These payload molecules are also referred to as modulatory nucleic acid payloads.

microRNAs (or miRNA) are 19-25 nucleotide long noncoding RNAs that bind to the 3′UTR of nucleic acid molecules and down-regulate gene expression either by reducing nucleic acid molecule stability or by inhibiting translation. The payload constructs described herein can include one or more microRNA target sequences, microRNA sequences, or microRNA seeds. Such sequences can correspond to any known microRNA such as those taught in US Publication US2005/0261218 and US Publication US2005/0059005, the contents of which are incorporated herein by reference in their entirety.

A microRNA sequence includes a seed region, i.e., a sequence in the region of positions 2-8 of the mature microRNA, which has perfect Watson-Crick complementarity to the miRNA target sequence. A microRNA seed can include positions 2-8 or 2-7 of the mature microRNA. In some embodiments, a microRNA seed can include 7 nucleotides (e.g., nucleotides 2-8 of the mature microRNA), wherein the seed-complementary site in the corresponding miRNA target is flanked by an adenine (A) opposed to microRNA position 1. In some embodiments, a microRNA seed can include 6 nucleotides (e.g., nucleotides 2-7 of the mature microRNA), wherein the seed-complementary site in the corresponding miRNA target is flanked by an adenine (A) opposed to microRNA position 1. See for example, Grimson A, Farh K K, Johnston W K, Garrett-Engele P, Lim L P, Bartel D P; Mol Cell. 2007 Jul. 6; 27(1):91-105; each of which is herein incorporated by reference in their entirety. The bases of the microRNA seed have complete complementarity with the target sequence.

A payload molecule can include proteins that serve as marker proteins to assess cell transformation and expression, fusion proteins, protein having a desired biological activity, gene products that can complement a genetic defect, RNA molecules, transcription factors, and other gene products that are of interest in regulation and/or expression. A payload molecule can include nucleotide sequences that provide a desired effect or regulatory function (e.g., transposons, transcription factors). A payload molecule can include, but is not limited to, hormone receptors (e.g., mineral corticosteroid, glucocorticoid, and thyroid hormone receptors); intramembrane proteins (e.g., TM-1 and TM-7); intracellular receptors (e.g., orphans, retinoids, vitamin D3 and vitamin A receptors); signaling molecules (e.g., kinases, transcription factors, or molecules such signal transducers and activators of transcription receptors of the cytokine superfamily (e.g. erythropoietin, growth hormone, interferons, and interleukins, and colony-stimulating factors); G-protein coupled receptors (e.g., receptors for hormones, calcitonin, epinephrine, gastrin, and paracrine or autocrine mediators, such as somatostatin or prostaglandins); neurotransmitter receptors (e.g., norepinephrine, dopamine, serotonin or acetylcholine); neurotransmitter producing enzymes (e.g., enzymes that produce dopamine or serotonin (e.g., aromatic 1-amino acid decarboxylase (AADC))); pathogenic antigens, which can be of viral, bacterial, allergenic, or cancerous origin; and tyrosine kinase receptors (e.g., insulin growth factor and nerve growth factor).

A payload molecule can include a gene therapy product. A gene therapy product can include a protein, an RNA molecule, or other gene product that, when expressed in a target cell, provides a desired therapeutic effect. In some embodiments, a gene therapy product can include a substitute for a non-functional gene that is absent or mutated. In some embodiments, a gene therapy product can include a method for elimination of a gene that is over-active or dysregulated. See e.g., Goldsmith et al., WO 90/07936, the contents of which are incorporated herein by reference in their entirety.

A payload construct encoding a payload molecule can include a selectable marker. A selectable marker can include a gene sequence or a protein encoded by that gene sequence expressed in a host cell that allows for the identification, selection, and/or purification of the host cell from a population of cells that can or cannot express the selectable marker. In one embodiment the selectable marker provides resistance to survive a selection process that would otherwise kill the host cell, such as treatment with an antibiotic. In some embodiments an antibiotic selectable marker can include one or more antibiotic resistance factors, including but not limited to, neomycin resistance (e.g., neo), hygromycin resistance, kanamycin resistance, and/or puromycin resistance.

In some embodiments a selectable marker can include a cell-surface marker, such as any protein expressed on the surface of the cell including, but not limited to, receptors, CD markers, lectins, integrins, or truncated versions thereof. In some embodiments, cells that include a cell-surface marker can be selected using an antibody targeted to the cell-surface marker. In some embodiments an antibody targeted to the cell-surface marker can be directly conjugated with a selection agent including, but not limited to, a fluorophore, sepharose, or magnetic bead. In some embodiments an antibody targeted to the cell-surface marker can be detected using a secondary labeled antibody or substrate which binds to the antibody targeted to the cell-surface marker. In some embodiments, a selectable marker can include negative selection by using an enzyme, including but not limited to, Herpes simplex virus thymidine kinase (HSVTK) that converts a pro-toxin (ganciclovir) into a toxin or bacterial Cytosine Deaminase (CD) which converts the pro-toxin 5′-fluorocytosine (5′-FC) into the toxin 5′-fluorouracil (5′-FU). In some embodiments, any nucleic acid sequence encoding a polypeptide can be used as a selectable marker including recognition by a specific antibody.

In some embodiments, a payload construct encoding a payload molecule can include a selectable marker including, but not limited to, β-lactamase, luciferase, β-galactosidase, or any other reporter gene as that term is understood in the art, including cell-surface markers, such as CD4 or the truncated nerve growth factor (NGFR) (for GFP, see WO 96/23810; Heim et al., Current Biology 2: 178-182 (1996); Heim et al., Proc. Natl. Acad. Sci. USA (1995); or Heim et al., Science 373:663-664 (1995); for β-lactamase, see WO 96/30540). In some embodiments, a nucleic acid encoding a selectable marker can include a fluorescent protein. A fluorescent protein as herein described can include any fluorescent marker including, but not limited to, green, yellow, and/or red fluorescent protein (GFP, YFP, and RFP).

In accordance with the disclosure, a payload molecule including a nucleic acid for expression in a target cell will be incorporated into the parvovirus particle produced in the viral replication cell if the payload molecule is located between two ITR sequences.

A payload construct sequence encoding one or more payload molecules for expression in a target cell can include one or more nucleotide sequences operably linked to at least one target cell-compatible promoter. A payload construct sequence can also include one or more enhancer region sequences, one or more intron within the coding region of a payload, and/or a polyadenylation signal sequence, which can be useful for regulating expression of the payload molecule. A person skilled in the art can recognize that a target cell can require a specific promoter, enhancer, intron or polyadenylation signal sequence, including, but not limited to, a promoter that is species specific, inducible, tissue-specific, or cell cycle-specific Parr et al., Nat. Med. 3:1145-9 (1997).

Additional, non-limiting examples of promoters that can be used in a payload construct include, but are not limited to, the cytomegalovirus (CMV) promoter (Kaplitt et al. (1994) Nat. Genet. 8:148-154), CMV/human β3-globin promoter (Mandel et al. (1998) J. Neurosci. 18:4271-4284), NCXI promoter, αMEC promoter, MLC2v promoter, GFAP promoter (Xu et al. (2001) Gene Ther., 8:1323-1332), the 1.8-kb neuron-specific enolase (NSE) promoter (Klein et al. (1998) Exp. Neurol. 150:183-194), chicken beta actin (CBA) promoter (Miyazaki (1989) Gene 79:269-277) and the β-glucuronidase (GUSB) promoter (Shipley et al. (1991) Genetics 10:1009-1018), the human serum albumin promoter, the alpha-1-antitrypsin promoter. To improve expression, other regulatory elements may additionally be operably linked to the transgene, such as, e.g., the Woodchuck Hepatitis Virus Post-Regulatory Element (WPRE) (Dorello et al. (1998) J. Virol. 72: 5085-5092), a CMV enhancer sequence, a human β-globin intron sequence, an immediate-early 1 intron sequence, the human β-globin polyadenylation signal sequence or the bovine growth hormone (BGH) polyadenylation signal sequence.

### IV. VIRAL PRODUCTION

### General

The production of parvovirus particles having a partial self-complementary parvovirus genome described herein includes methods for producing parvovirus particles that can contact a target cell to deliver a payload construct that includes a nucleotide encoding a payload molecule described herein. Accordingly, in some embodiments, the present disclosure provides a method for generation of partial self-complementary parvovirus genomes and parvovirus particles as described herein during parvovirus production in insect cells.

In some embodiments, the present disclosure provides a method of making a population of parvovirus (e.g., AAV) particles that can include: (a) culturing insect cells with plasmid vectors encoding the parvovirus genomes of the present disclosure; (b) culturing insect cells with the parvovirus genomes to produce a population of parvovirus particles described herein; and (c) harvesting the population of parvovirus particles produced by the insect cells, wherein the harvested population of parvovirus particles include parvovirus particles having the high molecular weight parvovirus genome that can include a partial self-complementary parvovirus genome described herein. In some embodiments, the population of parvovirus (e.g., AAV) particles produced by the method is enriched for the parvovirus particles that have the high molecular weight parvovirus genome that can include a partial self-complementary parvovirus genome described herein.

In some embodiments, the present disclosure provides a method for producing a population of parvovirus (e.g., AAV) particles having the partial self-complementary genome described herein by the steps of: (a) culturing insect cells; (b) infecting the insect cells with a first BIIC and a second BIIC, wherein the first BIIC includes a baculovirus expression vector including a nucleotide sequence that encodes a parvovirus genome described herein, and wherein the second BIIC includes a baculovirus expression vector including a nucleotide sequence that produces parvovirus non-structural and structural proteins necessary for parvovirus particle formation in the insect cells; and (c) harvesting the parvovirus particles produced by the insect cells following the infection step (b), wherein the harvested parvovirus particles include a population of parvovirus particles having a high molecular weight parvovirus (e.g., AAV) genome that can include a partial self-complementary parvovirus genome described herein. One exemplary process for performing such a method is described in Example 1.

In some embodiments, the method for producing a population of parvovirus particles having the high molecular weight parvovirus (e.g., AAV) genome that can include a partial self-complementary genome described herein can also include the step of enriching the parvovirus particles for sub-population of parvovirus particles each having the high molecular weight parvovirus (e.g., AAV) genome that can include a partial self-complementary genome described herein. Methods for enriching for the first sub-population of parvovirus particles as well known in the art, including the methods described in Example 2, which includes enriching for the first sub-subpopulation by use of density gradient centrifugation. In some aspects, the density gradient centrifugation can be isopycnic centrifugation.

### Cells

The present disclosure provides an insect cell that includes a high molecular weight parvovirus (e.g., AAV) genome that can include a partial self-complementary parvovirus genome described herein. Viral production disclosed herein describes processes and methods for producing parvovirus particles that have a partial self-complementary genome described herein. In some embodiments; the parvovirus particle described herein can be produced in a viral replication cell that includes an insect cell.

Growing conditions for insect cells in culture, and production of heterologous products in insect cells in culture are well-known in the art, see U.S. Pat. No. 6,204,059, the contents of which are herein incorporated by reference in their entirety.

Any insect cell which allows for replication of parvovirus and which can be maintained in culture can be used in accordance with the present disclosure. Cell lines can be used from Spodoptera frugiperda, including, but not limited to, the pupal ovarian Sf9 or Sf21 cell lines, drosophila cell lines, or mosquito cell lines, such as, Aedes albopictus derived cell lines. Use of insect cells for expression of heterologous proteins is well documented, as are methods of introducing nucleic acids, such as vectors, e.g., insect-cell compatible vectors, into such cells and methods of maintaining such cells in culture. See, for example, METHODS IN MOLECULAR BIOLOGY, ed. Richard, Humana Press, NJ (1995); O'Reilly et al., BACULOVIRUS EXPRESSION VECTORS, A LABORATORY MANUAL, Oxford Univ. Press (1994); Samulski et al., J. Vir. 63:3822-8 (1989); Kajigaya et al., Proc. Nat'l. Acad. Sci. USA 88: 4646-50 (1991); Ruffing et al., J. Vir. 66:6922-30 (1992); Kimbauer et al., Vir. 219:37-44 (1996); Zhao et al., Vir. 272:382-93 (2000); and Samulski et al., U.S. Pat. No. 6,204,059, the contents of each of which are herein incorporated by reference in their entirety.

### Production of the Parvovirus Particles Using Baculovirus

Baculovirus expression vectors for producing parvovirus particles in insect cells including, but not limited to, Spodoptera frugiperda (Sf9) cells, provide high titers of parvovirus particle product. Recombinant baculovirus encoding the viral construct expression vector and payload construct expression vector initiates a productive infection of viral replicating cells. Infectious baculovirus particles released from the primary infection secondarily infect additional cells in the culture, exponentially infecting the entire cell culture population in a number of infection cycles that is a function of the initial multiplicity of infection, see Urabe, M. et al. J Virol. 2006 February; 80(4):1874-85, the contents of which are herein incorporated by reference in their entirety.

Production of parvovirus particles with baculovirus in an insect cell system can address known baculovirus genetic and physical instability. In one embodiment, the production system provided herein addresses baculovirus instability over multiple passages by utilizing a titerless infected-cells preservation and scale-up system. Small scale seed cultures of viral producing cells are infected with viral expression constructs encoding the structural, non-structural, components of the parvovirus particle. Baculovirus-infected viral producing cells are harvested into aliquots that can be cryopreserved in liquid nitrogen; the aliquots retain viability and infectivity for infection of large scale viral producing cell culture Wasilko D J et al. Protein Expr Purif 2009 June; 65(2):122-32, the contents of which are herein incorporated by reference in their entirety.

A genetically stable baculovirus can be used to produce the source of one or more of the components for producing parvovirus particles in invertebrate cells. In one embodiment, defective baculovirus expression vectors can be maintained episomally in insect cells. In such an embodiment the bacmid vector is engineered with replication control elements including, but not limited to, promoters, enhancers, and/or cell-cycle regulated replication elements.

In some embodiments, baculoviruses can be engineered with a (non-) selectable marker for recombination into the chitinase/cathepsin locus. The chia/v-cath locus is non-essential for propagating baculovirus in tissue culture, and the V-cath (EC 3.4.22.50) is a cysteine endoprotease that is most active on Arg-Arg dipeptide containing substrates. The Arg-Arg dipeptide is present in densovirus and parvovirus capsid structural proteins but infrequently occurs in dependovirus VP1.

In some embodiments, stable viral replication cells permissive for baculovirus infection are engineered with at least one stable integrated copy of any of the elements necessary for AAV replication and parvovirus particle production including, but not limited to, the entire AAV genome, Rep and Cap genes, Rep genes, Cap genes, each Rep protein as a separate transcription cassette, each VP protein as a separate transcription cassette, the AAP (assembly activation protein), or at least one of the baculovirus helper genes with native or non-native promoters.

In some embodiments, large-scale viral production methods of the present disclosure can include the use of suspension cell cultures. Suspension cell culture allows for significantly increased numbers of cells. Typically, the number of adherent cells that can be grown on about 10-50 cm² of surface area can be grown in about 1 cm³ volume in suspension.

Transfection of replication cells in large-scale culture formats can be carried out according to any methods known in the art. For large-scale adherent cell cultures, transfection methods can include, but are not limited to, the use of inorganic compounds (e.g. calcium phosphate,) organic compounds [e.g. polyethyleneimine (PEI)] or the use of non-chemical methods (e.g. electroporation). With cells grown in suspension, transfection methods can include, but are not limited to the use of calcium phosphate and the use of PEI. In some cases, transfection of large-scale suspension cultures can be carried out according to the section entitled “Transfection Procedure” described in Feng, L. et al., 2008. Biotechnol Appl Biochem. 50:121-32, the contents of which are herein incorporated by reference in their entirety. According to such embodiments. PEI-DNA complexes can be formed for introduction of plasmids to be transfected. In some cases, cells being transfected with PEI-DNA complexes can be ‘shocked’ prior to transfection. This includes lowering cell culture temperatures to 4° C. for a period of about 1 hour. In some cases, cell cultures can be shocked for a period of from about 10 minutes to about 5 hours. In some cases, cell cultures can be shocked at a temperature of from about 0° C. to about 20° C.

In some cases, transfections can include one or more vectors for expression of an RNA effector molecule to reduce expression of nucleic acids from one or more payload construct. Such methods can enhance the production of parvovirus particles by reducing cellular resources wasted on expressing payload constructs. In some cases, such methods can be carried according to those taught in US Publication No. US2014/0099666, the contents of which are herein incorporated by reference in their entirety.

Cells described herein, including, but not limited to viral production cells, can be subjected to cell lysis according to any methods known in the art. Cell lysis can be carried out to obtain one or more agents (e.g. parvovirus particles) present within any cells described herein. In some embodiments, cell lysis can be carried out according to any of the methods listed in U.S. Pat. Nos. 7,326,555, 7,579,181, 7,048,920, 6,410,300, 6,436,394, 7,732,129, 7,510,875, 7,445,930, 6,726,907, 6,194,191, 7,125,706, 6,995,006, 6,676,935, 7,968,333, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508 or International Publication Nos. WO1996039530, WO1998010088, WO1999014354, WO1999015685, WO1999047691, WO2000055342, WO2000075353 and WO2001023597, the contents of each of which are herein incorporated. by reference in their entirety. Cell lysis methods can be chemical or mechanical. Chemical cell lysis typically includes contacting one or more cells with one or more lysis agents. Mechanical lysis typically includes subjecting one or more cells to one or more lysis conditions and/or one or more lysis forces.

In some embodiments, chemical lysis can be used to lyse cells. As used herein, the term lysis agent refers to any agent that can aid in the disruption of a cell. In some cases, lysis agents are introduced in solutions, termed lysis solutions or lysis buffers. As used herein, the term lysis solution refers to a solution (typically aqueous) including one or more lysis agents. In addition to lysis agents, lysis solutions can include one or more buffering agents, solubilizing agents, surfactants, preservatives, cryoprotectants, enzymes, enzyme inhibitors and/or chelators. Lysis buffers are lysis solutions including one or more buffering agents. Additional components of lysis solutions can include one or more solubilizing agents. As used herein, the term solubilizing agent refers to a compound that enhances the solubility of one or more components of a solution and/or the solubility of one or more entities to which solutions are applied. In some cases, solubilizing agents enhance protein solubility, In some cases, solubilizing agents are selected based on their ability to enhance protein solubility while maintaining protein conformation and/or activity.

Exemplary lysis agents can include any of those described in U.S. Pat. Nos. 8,685,734, 7,901,921, 7,732,129, 7,223,585, 7,125,706, 8,236,495, 8,110,351, 7,419,956, 7,300,797, 6,699,706 and 6,143,567, the contents of each of which are herein incorporated by reference in their entirety. In some cases, lysis agents can be selected from lysis salts, amphoteric agents, cationic agents, ionic detergents and non-ionic detergents. Lysis salts can include, but are not limited to, sodium chloride (NaCl) and potassium chloride (KCl). Further lysis salts can include any of those described in U.S. Pat. Nos. 8,614,101, 7,326,555, 7,579,181, 7,048,920, 6,410,300, 6,436,394, 7,732,129, 7,510,875, 7,445,930, 6,726,907, 6,194,191, 7,125,706, 6,995,006, 6,676,935 and 7,968,333, the contents of each of which are herein incorporated by reference in their entirety. Concentrations of salts can be increased or decreased to obtain an effective concentration for rupture of cell membranes. Amphoteric agents, as referred to herein, are compounds capable of reacting as an acid or a base. Amphoteric agents can include, but are not limited to, lysophosphatidylcholine, 3-((3-Cholamidopropyl)dimethylammonium)-1-propanesulfonate (CHAPS), ZWITTERGENT® and the like. Cationic agents can include, but are not limited to, cetyltrimethylammonium bromide (C(16)TAB) and Benzalkonium chloride. Lysis agents including detergents can include ionic detergents or non-ionic detergents. Detergents can function to break apart or dissolve cell structures including, but not limited to, cell membranes, cell walls, lipids, carbohydrates, lipoproteins and glycoproteins. Exemplary ionic detergents include any of those taught in U.S. Pat. Nos. 7,625,570 and 6,593,123 or US Publication No. US2014/0087361, the contents of each of which are herein incorporated by reference in their entirety. Some ionic detergents can include, but are not limited to, sodium dodecyl sulfate (SDS), cholate and deoxycholate. In some cases, ionic detergents can be included in lysis solutions as a solubilizing agent. Non-ionic detergents can include, but are not limited to, octylglucoside, digitonin, lubrol, C12E8, TWEEN®-20, TWEEN®-80, Triton X-100 and Noniodet P-40. Non-ionic detergents are typically weaker lysis agents but can be included as solubilizing agents for solubilizing cellular and/or viral proteins. Further lysis agents can include enzymes and urea. In some cases, one or more lysis agents can be combined in a lysis solution in order to enhance one or more of cell lysis and protein solubility. In some cases, enzyme inhibitors can be included in lysis solutions in order to prevent proteolysis that can be triggered by cell membrane disruption.

In some embodiments, mechanical cell lysis is carried out. Mechanical cell lysis methods can include the use of one or more lysis conditions and/or one or more lysis forces. As used herein, the term lysis condition refers to a state or circumstance that promotes cellular disruption. Lysis conditions can include certain temperatures, pressures, osmotic purity, salinity and the like. In some cases, lysis conditions include increased or decreased temperatures. According to some embodiments, lysis conditions include changes in temperature to promote cellular disruption. Cell lysis carried out according to such embodiments can include freeze-thaw lysis. As used herein, the term freeze-thaw lysis refers to cellular lysis in which a cell solution is subjected to one or more freeze-thaw cycles. According to freeze-thaw lysis methods, cells in solution are frozen to induce a mechanical disruption of cellular membranes caused by the formation and expansion of ice crystals. Cell solutions used according to freeze-thaw lysis methods, can further include one or more lysis agents, solubilizing agents, buffering agents, cryoprotectants, surfactants, preservatives, enzymes, enzyme inhibitors and/or chelators. Once cell solutions subjected to freezing are thawed, such components can enhance the recovery of desired cellular products. In some cases, one or more cyroprotectants are included in cell solutions undergoing freeze-thaw lysis. As used herein, the term “cryoprotectant” refers to an agent used to protect one or more substances from damage due to freezing. Cryoprotectants described herein can include any of those taught in US Publication No. US201310323302 or U.S. Pat. Nos. 6,503,888, 6,180,613, 7,888,096, 7,091,030, the contents of each of which are herein incorporated by reference in their entirety. In some cases, cryoprotectants can include, but are not limited to dimethyl sulfoxide, 1,2-propanediol, 2,3-butanediol, formamide, glycerol, ethylene glycol, 1,3-propanediol and n-dimethyl formamide, polyvinylpyrrolidone, hydroxyethyl starch, agarose, dextrans, inositol, glucose, hydroxyethylstarch, lactose, sorbitol, methyl glucose, sucrose and urea. In some embodiments, freeze-thaw lysis can be carried out according to any of the methods described in U.S. Pat. No. 7,704,721, the contents of which are herein incorporated by reference in their entirety.

As used herein, the term lysis force refers to a physical activity used to disrupt a cell. Lysis forces can include, but are not limited to, mechanical forces, some threes, gravitational forces, optical forces, electrical forces and the like. Cell lysis carried out by mechanical three is referred to herein as mechanical lysis. Mechanical forces that can be used according to mechanical lysis can include high shear fluid forces. According to such methods of mechanical lysis, a microfluidizer can be used. Microfluidizers typically include an inlet reservoirs where cell solutions can be applied. Cell solutions can then be pumped into an interaction chamber via a pump (e.g. high-pressure pump) at high speed and/or pressure to produce shear fluid forces. Resulting lysates can then be collected in one or more output reservoir. Pump speed and/or pressure can be adjusted to modulate cell lysis and enhance recovery of products (e.g. parvovirus particles). Other mechanical lysis methods can include physical disruption of cells by scraping.

Cell lysis methods can be selected based on the cell culture format of cells to he lysed. For example, with adherent cell cultures, some chemical and mechanical lysis methods can be used. Such mechanical lysis methods can include freeze-thaw lysis or scraping. In another example, chemical lysis of adherent cell cultures can be carried out through incubation with lysis solutions including surfactant, such as Triton-X-100. In some cases, cell lysates generated from adherent cell cultures can be treated with one more nucleases to lower the viscosity of the lysates caused by liberated DNA.

### Clarification

Cell lysates including parvovirus particles can be subjected to clarification. Clarification refers to initial steps taken in purification of parvovirus particles from cell lysates. Clarification serves to prepare lysates for further purification by removing larger, insoluble debris. Clarification steps can include, but are not limited to, centrifugation and filtration. During clarification, centrifugation can be carried out at low speeds to remove larger debris, only. Similarly, filtration can be carried out using filters with larger pore sizes so that only larger debris is removed. In some cases, tangential flow filtration can be used during clarification. Objectives of viral clarification include high throughput processing of cell lysates and to optimize ultimate viral recovery. Advantages of including a clarification step include scalability for processing of larger volumes of lysate. In some embodiments, clarification can be carried out according to any of the methods presented in U.S. Pat. Nos. 8,524,446, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498, 7,491,508, US Publication Nos. US2013/0045186, US2011/0263027, US2011/0151434, US2003/0138772, and International Publication Nos. WO2002012455, WO1996039530, WO1998010088, WO1999014354, WO1999015685, WO1999047691, WO2000055342, WO2000075353 and WO2001023597, the contents of each of which are herein incorporated by reference in their entirety.

Methods of cell lysate clarification by filtration are well understood in the art and can be carried out according to a variety of available methods including, but not limited to, passive filtration and flow filtration. Filters used can include a variety of materials and pore sizes. For example, cell lysate filters can include pore sizes of from about 1 μM to about 5 μM, from about 0.5 μM to about 2 μM, from about 0.1 μM to about 1 μM, from about 0.05 μM to about 0.5 μM and from about 0.001 μM to about 0.1 μM. Exemplary pore sizes for cell lysate filters can include, but are not limited to, 2.0, 1.9, 1.8, 1.7, 1.6, 1.5, 1.4, 1.3, 1.2, 1.1, 1, 0.9, 0.8, 0.7, 0.6, 0.5, 0.4, 0.3, 0.2, 0.1, 0.95, 0.9, 0.85, 0.8, 0.75, 0.7, 0.65, 0.6, 0.55, 0.5, 0.45, 0.4, 0.35, 0.3, 0.25, 0.2, 0.15, 0.1, 0.05, 0.22, 0.21, 0.20, 0.19, 0.18, 0.17, 0.16, 0.15, 0.14, 0.13, 0.12, 0.11, 0.1, 0.09, 0.08, 0.07, 0.06, 0.05, 0.04, 0.03, 0.02, 0.01, 0.02, 0.019, 0.018, 0.017, 0.016, 0.015, 0.014, 0.013, 0.012, 0.011, 0.01, 0.009, 0.008, 0.007, 0.006, 0.005, 0.004, 0.003, 0.002, 0.001 and 0.001 μM. In one embodiment, clarification can include filtration through a filter with 2.0 μM pore size to remove large debris, followed by passage through a filter with 0.45 μM pore size to remove intact cells.

Filter materials can be composed of a variety of materials. Such materials can include, but are not limited to, polymeric materials and metal materials (e.g. sintered metal and pored aluminum). Exemplary materials can include, but are not limited to, nylon, cellulose materials (e.g. cellulose acetate), polyvinylidene fluoride (PVDF), polyethersulfone, polyamide, polysulfone, polypropylene and polyethylene terephthalate. In some cases, filters useful for clarification of cell lysates can include, but are not limited to, ULTIPLEAT PROFILE™ filters (Pall Corporation, Port Washington, N.Y.), SUPOR™ membrane filters (Pall Corporation, Port Washington, N.Y.)

In some cases, flow filtration can be carried out to increase filtration speed and/or effectiveness. In some cases, flow filtration can include vacuum filtration. According to such methods, a vacuum is created on the side of the filter opposite that of cell lysate to be filtered. In some cases, cell lysates can be passed through filters by centrifugal forces. In some cases, a pump is used to force cell lysate through clarification filters. Flow rate of cell lysate through one or more filters can be modulated by adjusting one of channel size and/or fluid pressure.

According to some embodiments, cell lysates can be clarified by centrifugation. Centrifugation can be used to pellet insoluble particles in the lysate. During clarification, centrifugation strength [expressed in terms of gravitational units (g), which represents multiples of standard gravitational force] can be lower than in subsequent purification steps. In some cases, centrifugation can be carried out on cell lysates at from about 200 g to about 800 g, from about 500 g to about 1500 g, from about 1000 g to about 5000 g, from about 1200 g to about 10000 g or from about 8000 g to about 15000 g. In some embodiments, cell lysate centrifugation is carried out at 8000 g for 15 minutes. In some cases, density gradient centrifugation can be carried out in order to partition particulates in the cell lysate by sedimentation rate. Gradients used according to methods of the present disclosure can include, but are not limited to, cesium chloride gradients and iodixanol step gradients.

### Purification—Chromatography

In some cases, parvovirus particles can be purified from clarified cell lysates by one or more methods of chromatography. Chromatography refers to any number of methods known in the art for separating out one or more elements from a mixture. Such methods can include, but are not limited to, ion exchange chromatography (e.g. cation exchange chromatography and anion exchange chromatography,) immunoaffinity chromatography and size-exclusion chromatography. In some embodiments, methods of viral chromatography can include any of those taught in U.S. Pat. Nos. 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508 or International Publication Nos. WO1996039530, WO1998010088, WO1999014354, WO1999015685, WO1999047691, WO2000055342, WO2000075353 and WO2001023597, the contents of each of which are herein incorporated by reference by reference in their entirety.

In some embodiments, ion exchange chromatography can be used to isolate parvovirus particles. Ion exchange chromatography is used to bind parvovirus particles based on charge-charge interactions between capsid proteins and charged sites present on a stationary phase, typically a column through which viral preparations (e.g. clarified lysates) are passed, After application of viral preparations, bound parvovirus particles can then be eluted by applying an elution solution to disrupt the charge-charge interactions. Elution solutions can be optimized by adjusting salt concentration and/or pH to enhance recovery of bound parvovirus particles. Depending on the charge of viral capsids being isolated, cation or anion exchange chromatography methods can be selected. Methods of ion exchange chromatography can include, but are not limited to, any of those taught in U.S. Pat. Nos. 7,419,817, 6,143,548, 7,094,604, 6,593,123, 7,015,026 and 8,137,948, the contents of each of which are herein incorporated by reference in their entirety.

In some embodiments, immunoaffinity chromatography can be used. Immunoaffinity chromatography is a form of chromatography that utilizes one or more immune compounds (e.g., antibodies or antibody-related structures) to retain parvovirus particles. Immune compounds can bind specifically to one or more structures on parvovirus particle surfaces, including, but not limited to, one or more viral coat proteins. In some cases, immune compounds can be specific for a particular viral variant. In some cases, immune compounds can bind to multiple viral variants. In some embodiments, immune compounds can include recombinant single-chain antibodies. Such recombinant single chain antibodies can include those described in Smith, R. H. et al., 2009. Mol Ther. 17(11):1888-96, the contents of which are herein incorporated by reference in their entirety. Such immune compounds are capable of binding to several AAV capsid variants, including, but not limited to, AAV1, AAV2, AAV6 and AAV8.

In some embodiments, size-exclusion chromatography (SEC) can be used. SEC can include the use of a gel to separate particles according to size. In parvovirus particle purification, SEC filtration is sometimes referred to as “polishing.” In some cases, SEC can be carried out to generate a final product that is near-homogenous. Such final products can in some cases be used in pre-clinical studies and/or clinical studies (Kotin, R. M. 2011. Human Molecular Genetics. 20(1):R2-R6, the contents of which are herein incorporated by reference in their entirety). In some cases, SEC can be carried out according to any of the methods taught in U.S. Pat. Nos. 6,143,548, 7,015,026, 8,476,418, 6,410,300, 8,476,418, 7,419,817, 7,094,604, 6,593,123, and 8,137,948, the contents of each of which are herein incorporated by reference in their entirety.

In one embodiment, the compositions including at least one parvovirus particle can be isolated or purified using the methods described in U.S. Pat. No. 6,146,874, the contents of which are herein incorporated by reference in their entirety.

In one embodiment, the compositions including at least one parvovirus particle can be isolated or purified using the methods described in U.S. Pat. No. 6,660,514, the contents of which are herein incorporated by reference in their entirety.

In one embodiment, the compositions including at least one parvovirus particle can be isolated or purified using the methods described in U.S. Pat. No. 8,283,151, the contents of which are herein incorporated by reference in their entirety.

In one embodiment, the compositions including at least one parvovirus particle can be isolated or purified using the methods described in U.S. Pat. No. 8,524,446, the contents of which are herein incorporated by reference in their entirety.

### Enrichment

In some embodiments, a population of parvovirus particles described herein and/or produced by a method described herein is enriched for parvovirus particles each having the high molecular weight parvovirus genome that can include a partial self-complementary parvovirus genome described herein, relative to a starting population. In some embodiments, a population of parvovirus particles described herein and/or produced by a method described herein is enriched for parvovirus particles having the low molecular weight parvovirus genome that can include a genome that does not include the nucleotide sequence that is complementary to a portion of the payload construct, relative to a starting population. A variety of enrichment procedures are available, including those that separate viral particles on the basis of molecular weight or differences in charge. Non-limiting examples of gradients for separating viral particles on the basis of molecular weight include isopycnic centrifugation in cesium chloride gradients or iodixanol step gradients.

Additionally, a non-limiting example of enriching based on a difference in charge includes anion exchange. In one example; fractions are collected following a progressive mix of low salt and high salt buffers, generating a salt gradient. Elution can be monitored by UV absorption at 260 and 280 nm. Using an anion exchanger, protein peaks from the lower salt eluate contain empty capsids, with viral particles containing higher molecular weight DNA being eluted at progressively higher salt concentrations. In some embodiments, anion exchange is performed using fast performance liquid chromatography.

In some embodiments, buffers for use with the anion exchange columns are cationic or zwitterionic in nature. Such buffers include, without limitation, buffers with the following buffer ions: N-methylpiperazine; piperazine; Bis-Tris; Bis-Tris propane; Triethanolamine; Tris; N-methyldiethanolamine; 1,3-diaminopropane; ethanolamine; acetic acid, and the like. To elute the sample; the ionic strength of the starting buffer is increased using a salt, such as NaCl, KCl, sulfate, formate or acetate, at an appropriate pH. In some embodiments, buffers used during, before or after anion-exchange chromatography comprise a non-ionic surfactanct, for example Pluronic® F-68 (ThermoFisher Scientific), in an amount ranging from 0.0001% to 0.1% (v/v) of the total volume of the buffer composition; which includes in an amount ranging from 0.0005% to 0.005% (v/v) of the total volume of the buffer composition; which includes about 0.001% (v/v) of the total volume of the buffer composition.

The nature of the resins used (i.e. strong or weak ion exchangers) and the conditions of salt concentration, buffer used, and pH, will vary on the viral capsid variant (e.g., AAV capsid serotype or pseudotype). Further non-limiting examples of anion-exchange for use in separating viral particles that differ in terms of amount of packaged DNA are provided in U.S. Pat. No. 7,261,544, WO2016128408, and WO2017160360; all of which are incorporated herein by reference.

### V. PHARMACEUTICAL COMPOSITIONS

The present disclosure provides a pharmaceutical composition including a parvovirus particle having a high molecular weight parvovirus (e.g., AAV) genome that can include a partial self-complementary parvovirus genome described herein and a pharmaceutically acceptable carrier.

The present disclosure also provides a pharmaceutical composition including a population of parvovirus particles described. herein and a pharmaceutically acceptable carrier.

Although the descriptions of pharmaceutical compositions, e.g., those viral vectors including a payload to be delivered, provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including murines, rats, rabbits, simians, bovines, ovines, porcines, canines, felines; farm animals, sport animals, pets, and equines.

In some embodiments, compositions are administered to humans, human patients or subjects. For the purposes of the present disclosure, the phrase active ingredient generally refers either to the parvovirus particle carrying the payload or to the payload molecule delivered by the parvovirus particle as described herein.

Formulations of the pharmaceutical compositions described herein can be prepared by any method known or hereafter developed in the art of pharmacology. In general, such preparatory methods include the step of bringing the active ingredient into association with a carrier and/or one or more other accessory ingredients, and then, if necessary and/or desirable, dividing, shaping and/or packaging the product into a desired single- or multi-dose unit.

Relative amounts of the active ingredient, the pharmaceutically acceptable carrier; and/or any additional ingredients in a pharmaceutical composition in accordance with the disclosure will vary, depending upon the identity, size, and/or condition of the subject treated and further depending upon the route by which the composition is to be administered.

### Formulation

The parvovirus particles described herein can be formulated using one or more carriers, excipients, stabilizers and adjuvants to, for example: (1) increase stability; (2) increase cell transfection or transduction; (3) permit the sustained or delayed release; (4) alter the biodistribution (e.g., target the parvovirus particle to specific tissues or cell types); (5) increase the translation of encoded protein in vivo; and/or (6) alter the release profile of encoded protein in vivo.

Formulations of the pharmaceutical compositions provided herein can include, without limitation, saline, lipidoids, liposomes, lipid nanoparticles, polymers, lipoplexes, core-shell nanoparticles, peptides, proteins, cells infected with viral vectors (e.g., for transplantation into a subject), nanoparticle mimics and combinations thereof. Further, the parvovirus particles disclosed herein can be formulated using self-assembled nucleic acid nanoparticles.

Formulations of the pharmaceutical compositions described herein can be prepared by any method known or hereafter developed in the art of pharmacology. In general, such preparatory methods include the step of associating the active ingredient with a carrier and/or one or more other accessory ingredients (e.g., excipients, stabilizers and adjuvants).

A pharmaceutical composition in accordance with the present disclosure can be prepared, packaged, and/or sold in bulk, as a single unit dose, and/or as a plurality of single unit doses. As used herein, a unit dose refers to a discrete amount of the pharmaceutical composition including a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient (e.g. parvovirus particle), the pharmaceutically acceptable carrier, and/or any additional ingredients in a pharmaceutical composition in accordance with the present disclosure can vary, depending upon the identity, size, and/or condition of the subject being treated and further depending upon the route by which the composition is to be administered. For example, the composition can include between 0.1% and 99% (w/w) of the active ingredient. By way of example, the composition can include between 0.1% and 99%, e.g., between 0.5 and 50%, between 1-30%, between 5-80%, at least 80% (w/w) active ingredient.

In some embodiments, the formulations described herein can contain at least one parvovirus population. As a non-limiting example, the formulations can contain 1, 2, 3, 4 or 5 parvovirus populations. In one embodiment the formulation can contain a parvovirus particle having a payload construct encoding proteins selected from categories such as, but not limited to, human proteins, veterinary proteins, bacterial proteins, biological proteins, antibodies, immunogenic proteins, therapeutic peptides and proteins, secreted proteins, plasma membrane proteins, cytoplasmic and cytoskeletal proteins, intracellular membrane bound proteins, nuclear proteins, proteins associated with human disease and/or proteins associated with non-human diseases. In one embodiment, the formulation contains at least three parvovirus populations encoding proteins.

The formulations described herein can include one or more carriers, excipients, stabilizers and adjuvants, each in an amount that together, for example, increases the stability of the parvovirus particle, increases cell transfection or transduction by the parvovirus particle, increases the expression of parvovirus particle encoded protein, and/or alters the release profile of parvovirus particle encoded proteins. In some embodiments, a pharmaceutically acceptable excipient can be at least 95%, at least 96%, at least 97%, at least 98%, at least 99%, or 100% pure. In some embodiments, an excipient is approved for use for humans and for veterinary use. In some embodiments, an excipient can be approved by United States Food and Drug Administration. In some embodiments, an excipient can be of pharmaceutical grade. In some embodiments, an excipient can meet the standards of the United States Pharmacopoeia (USP), the European Pharmacopoeia (EP), the British Pharmacopoeia, and/or the International Pharmacopoeia.

Various carriers, excipients, stabilizers and adjuvants for formulating pharmaceutical compositions and techniques for preparing the composition are known in the art (see Remington: The Science and Practice of Pharmacy, 22nd Revised Ed., Pharmaceutical Press, 2012; incorporated herein by reference in its entirety). The use of a conventional carriers, excipients, stabilizers and adjuvants can be contemplated within the scope of the present disclosure, except insofar as any conventional excipient medium can be incompatible with a substance or its derivatives, such as by producing any undesirable biological effect or otherwise interacting in a deleterious manner with any other component(s) of the pharmaceutical composition.

### VI. KITS AND DEVICES

Also provided herein are a variety of kits for conveniently and/or effectively carrying out methods of the present disclosure. Typically, kits will include sufficient amounts and/or numbers of components to allow a user to perform multiple treatments of a subject(s) and/or to perform multiple experiments.

In one aspect, the present disclosure provides kits including the molecules (parvovirus particles) described herein. In one embodiment, the kit includes one or more parvirus particle or population thereof.

Said kits can be for parvovirus particle administration. The kit can further include packaging and instructions and/or a delivery agent to form a formulation composition. The delivery agent can include a saline, a buffered solution, or any delivery agent disclosed herein.

In some embodiments, kit components can be packaged either in aqueous media or in lyophilized form. The container means of the kits will generally include at least one vial, test tube, flask, bottle, syringe or other container means, into which a component can be placed, and preferably, suitably aliquoted. Where there are more than one kit component, (labeling reagent and label can be packaged together), kits can also generally contain second, third or other additional containers into which additional components can be separately placed. In some embodiments, kits can also include second container means for containing sterile, pharmaceutically acceptable buffers and/or other diluents. In some embodiments, various combinations of components can be included in one or more vial. Kits of the present disclosure can also typically include means for containing compounds and/or compositions of the present disclosure particles), and any other reagent containers in close confinement for commercial sale. Such containers can include injection or blow-molded plastic containers into which desired vials are retained.

In some embodiments, kit components are provided in one and/or more liquid solutions. In some embodiments, liquid solutions are aqueous solutions, with sterile aqueous solutions being particularly preferred. In some embodiments, kit components can be provided as dried powder(s). When reagents and/or components are provided as dry powders, such powders can be reconstituted by the addition of suitable volumes of solvent. In some embodiments, it is envisioned that solvents can also be provided in another container means.

In some embodiments, kits can include instructions for employing kit components as well the use of any other reagent not included in the kit. Instructions can include variations that can be implemented.

In some embodiments, compounds and/or compositions of the present disclosure can be combined with, coated onto or embedded in a device. Devices can include, but are not limited to, dental implants, stents, bone replacements, artificial joints, valves, pacemakers and/or other implantable therapeutic devices.

The present disclosure provides for devices which can incorporate parvovirus particles that encode one or more payload molecules. These devices contain in a stable formulation the parvovirus particles which can be immediately delivered to a subject in need thereof, such as a human patient.

Devices for administration can be employed to deliver the parvovirus particles of the present disclosure according to single, multi- or split-dosing regimens taught herein.

Method and devices known in the art for multi-administration to cells, organs and tissues are contemplated for use in conjunction with the methods and compositions disclosed herein as embodiments of the present disclosure. These include, for example, those methods and devices having multiple needles, hybrid devices employing for example lumens or catheters as well as devices utilizing heat, electric current or radiation driven mechanisms.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or the entire group members are present in, employed in, or otherwise relevant to a given product or process.

It is also noted that the term “comprising,” “containing,” “including,” or “having” is intended to be open and permits but does not require the inclusion of additional elements or steps. When the term “comprising,” “containing,” “including,” or “having is used herein, the term “consisting of” is thus also encompassed and disclosed.

It is understood that modifications which do not substantially affect the activity of the various embodiments of this disclosure are also provided within the definition of the invention provided herein. Accordingly, the following examples are intended to illustrate but not limit the present invention.

### VII. EXAMPLES

### Example 1

### Production and Isolation of AAV Particles

A population of AAV particles was produced utilizing an Sf9/Baculovirus production system, with Sf9 cells cultured in serum-free media. The transgene packaged included a sequence encoding human aromatic L-amino acid decarboxylase (hAADC), with a total insert size of about 3200 bases (total monomer size of about 3500 bases, including the two ITRs). An expression cassette containing the hAADC transgene was transposed into a baculovirus shuttle vector (bacmid) propagated in E. coli. A second bacmid containing an expression cassette containing rep2/cap2 transgenes was similarly prepared. Sf9 insect cells were infected with bacmid DNAs to produce baculovirus expression vectors (BEVs). Sf9 cells were then infected with a BEV, and the infected cells were frozen and banked as baculovirus infected insect cells (BACs), one encoding the hAADC expression cassette (BIIC-hAADC) and one encoding the AAV2 rep and cap genes (BIIC-rep2/cap2). BACs were used to infect Sf9 cells to generate recombinant AAV2-packaged hAADC (rAAV2-hAADC.

To generate the rAAV2-hAADC vector particles, Sf9 cells were initially seeded into flasks, then cells were passaged into progressively larger shaker flasks, then bags and finally into a bioreactor. Cells were infected with the two BIICs, followed by cell lysis, harvesting and then clarification by filtration. rAAV2-hAADC particles were then purified using two orthogonal purification steps, sepharose and cation exchange. The resulting solution from the cation exchange column was subjected to buffer exchange and concentration by ultrafiltration/diafiltration (UF/DF), then nanofiltration to reduce aggregated material and potential for other large impurities. The resulting product was sterile-filtered before being filled into glass vials. The resulting vector exhibited a ratio of full versus empty capsids of about 60%.

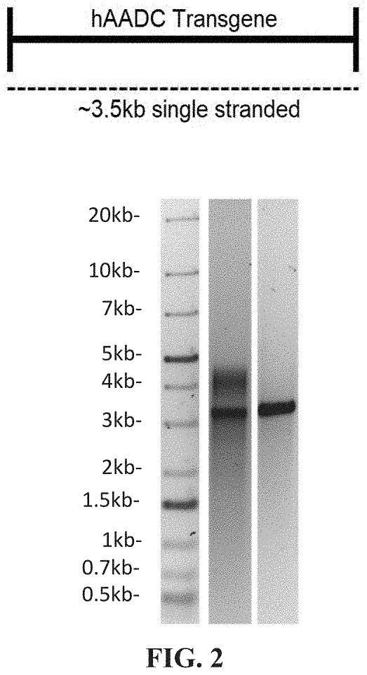

Packaged genomes from a sample of the produced rAAV2-hAADC were analyzed on an alkaline denaturing gel. Whereas rAAV2 packaged using human HEK293 cells yielded a single hand, the rAAV2-hAADC yielded two bands, one at about 3.5 kb (referred to as the “Low molecular weight” (Low MW) band) and one at about 4.5-4.7 kb (referred to as the “High molecular weight” (High MW) band). An illustrative gel image appears in FIG. 2, with lanes from left to right of molecular marker, Sf9-produced rAAV2-hAADC, and HEK293-produced rAAV2-hAADC. As noted above, the band at about 3.5 kb corresponds to the size of the hAADC expression cassette plus the flanking ITRs.

### Example 2

### Characterization of High MW Band

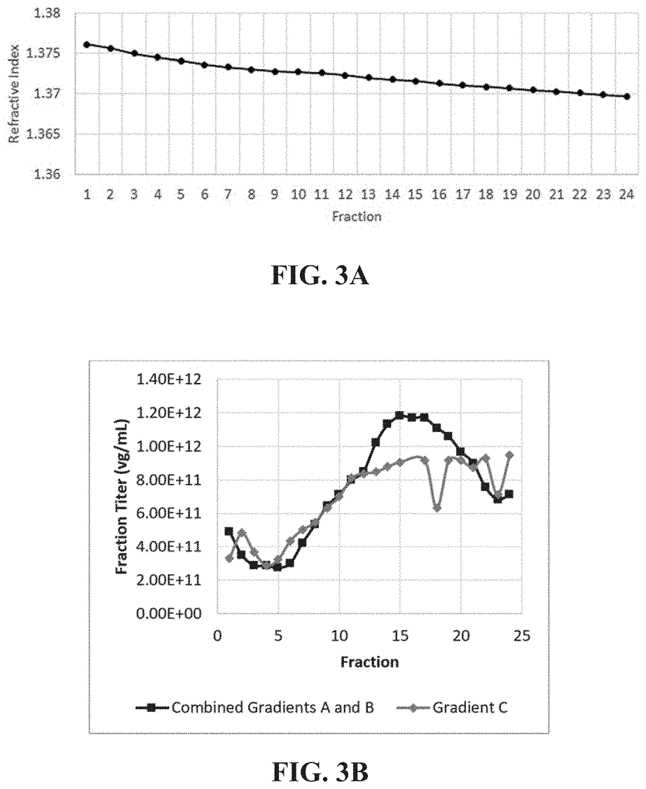

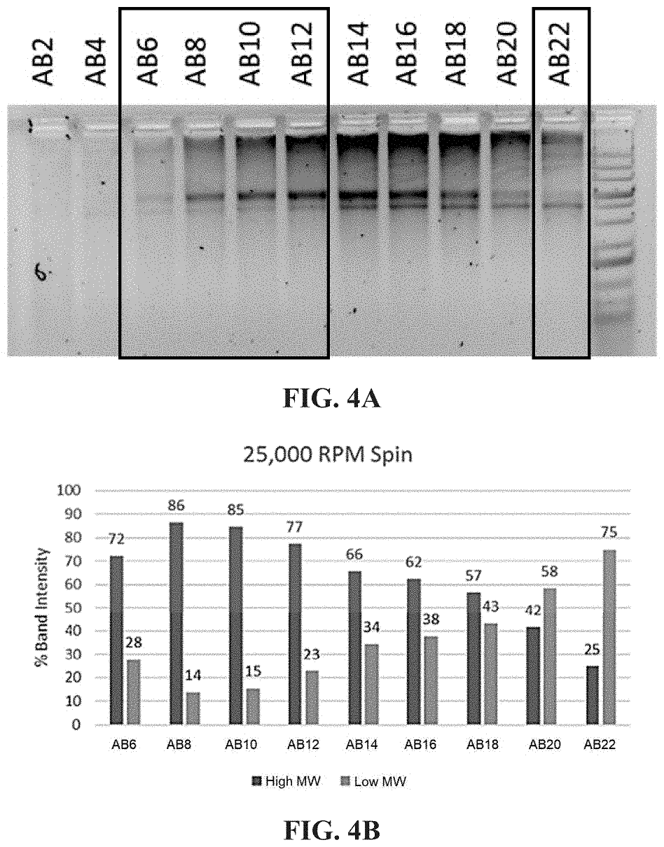

The rAAV2-hAADC population comprising high NTW and low MW sub-populations produced as in Example 1 was added to three CsCl isopycnic gradients (approximately 9×10¹² viral genomes (vg) per gradient). The gradients were all nm at a starting CsCl refractive index of approximately 1.372. Two of the gradients were spun in an ultracentrifuge at 25,000 rpm for 15 hours at 17° C. The third gradient was spun at 20,000 rpm for 15 hours at 17° C. The gradients were fractionated by dripping from the bottom of the gradient to generate 24 fractions of about 0.5 mL. The gradients were all linear between fractions 5 to 24. An example plot of the refractive indices of viral fractions is shown in FIG. 3A. The first two gradients (gradients A and B) were very similar in refractive indices, and corresponding fractions were combined (i.e., fraction 1 from both A and B were combined to generate fraction “AB1;” fraction 2 from both A and B were combined to generate fraction “AB2;” etc.). A small aliquot of the fractions was dialyzed and tested for qPCR titer, results for which are illustrated in FIG. 3B. Both data sets had titers between 2×10¹¹ vg/mL and 1.2×10¹² vg/mL for the different fractions. The dialyzed fractions were also nm on an alkaline denaturing gel, an illustrative image of which is shown in FIG. 4A. The two populations of rAAV2-hAADC, those with the High MW form and those with the Low MW form, were not separated entirely, but FIG. 4A shows that there was an enrichment for the High MW form in the heavier fractions. Relative quantities of the two populations was measured using densitometry. FIG. 4B shows a plot of relative proportions of High MW and Low MW forms (left and right bar in each pair, respectively) for each of the indicated fractions.

Fractions AB6 to AB12, all enriched for the High MW form, were combined (collectively referred to in this example as “High MW AADC”). Fractions AB22 to AB24, all enriched for the Low MW form, were combined (collectively referred to in this example as “Low MW AADC”). Although AB23 and AB24 were not analyzed on the denaturing gel, the population was likely similar to that of AB22, and both had a similar titer to AB22. Gradient C did not show much separation of the two DNA populations and was not used further. The High MW AADC and Low MW AADC were dialyzed overnight against buffer. The dialyzed samples were titered by qPCR. The average titer for the High MW AADC was 6.64×10¹¹ vg/mL (1.44% CV). The average titer for the Low MW AADC was 8.08×10¹¹ vg/mL (0.39% CV).

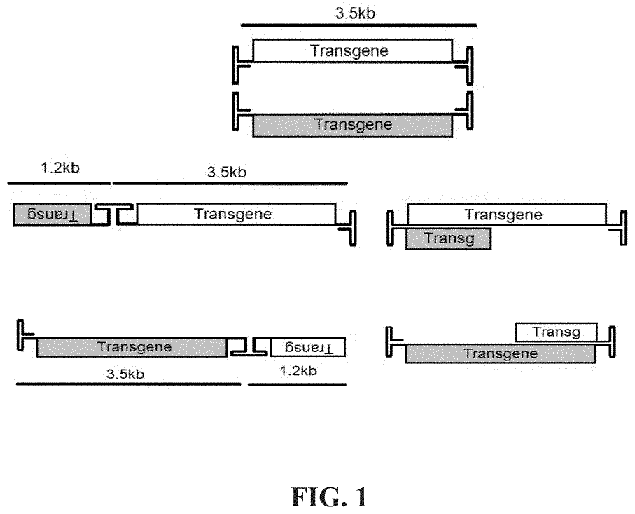

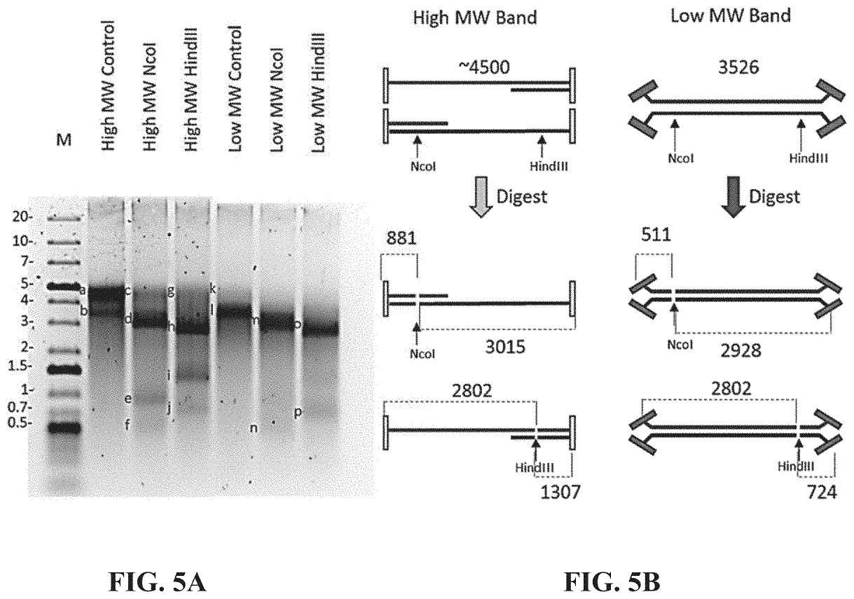

A portion (1.5 mL) of the dialyzed materials was digested with proteinase K followed by heat denaturation. The digests were then purified using QIAGEN QIAquick PCR Cleanup Kit. The extracted DNA was then digested with either NcoI or HindIII endonuclease. The digested DNA was analyzed by denaturing gel. FIG. 5A shows an image of the denaturing gel, and FIG. 5B shows illustrations of the predicted genomic structures and digestion products. Table 1 below shows fragment sizes predicted based on the predicted genomic structures and the fragment sizes observed on the gel. Observed fragments are grouped by gel lane of FIG. 5A, and within each lane, listed in order from largest to smallest, and designated (a) to (p). The fragment sizes were in agreement with those based on the predicted structures illustrated in FIG. 5B. A further illustration of the High MW and Low MW genome structures indicated by these digestion results is shown in FIG. 1, which shows the partially self-complementary encapsidated DNA formed by packaging a full copy of the transgene flanked by ITRs in addition to a portion of the transgene extending beyond one of the ITRs, such that one ITR is flanked by transgene sequences.

TABLE 1

| | Predicted | Observed | | Fragment Description |
|---|---|---|---|---|
| High MW | ~4500 | ~4480 | (a) | High molecular weight transgene |
| Control | | | | DNA |
| | ~3530 | ~3340 | (b) | Low molecular weight transgene |
| | | | | DNA (low propostion) |
| High MW | ~4500 | ~4330 | (c) | Residual High MW fragment |
| NcoI | | | | from opposite strand polarity |
| | ~3000 | ~2960 | (d) | Large piece resulting from |
| | | | | digestion |
| | ~880 | ~950 | (e) | Hairpin piece resulting from |
| | | | | digestion |
| | ~510 | ~500 | (f) | Digested piece from Low MW |
| | | | | transgene (low proportion) |
| High MW | ~4500 | ~4330 | (g) | Residual High MW fragment |
| HindIII | | | | from opposite strand polarity |
| | ~2800 | ~2930 | (h) | Large piece resulting from |
| | | | | digestion |
| | ~1310 | ~1430 | (i) | Hairpin piece resulting from |
| | | | | digestion |
| | ~720 | ~800 | (j) | Digested piece from Low MW |
| | | | | transgene (low proportion) |
| Low MW | ~4500 | ~4760 | (k) | High molecular weight transgene |
| Control | | | | DNA (low proportion) |
| | ~3530 | ~3530 | (l) | Low molecular weight transgene |
| | | | | DNA |
| Low MW | ~2930 | ~3130 | (m) | Large piece resulting from |
| NcoI | | | | digestion |
| | ~510 | ~560 | (n) | Small piece resulting from |
| | | | | digestion |
| Low MW | ~2800 | ~2950 | (o) | Large piece resulting from |
| HindIII | | | | digestion |
| | ~720 | ~740 | (p) | Small piece resulting from |
| | | | | digestion |

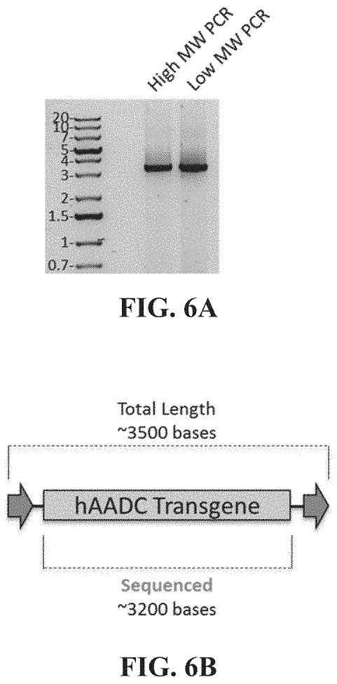

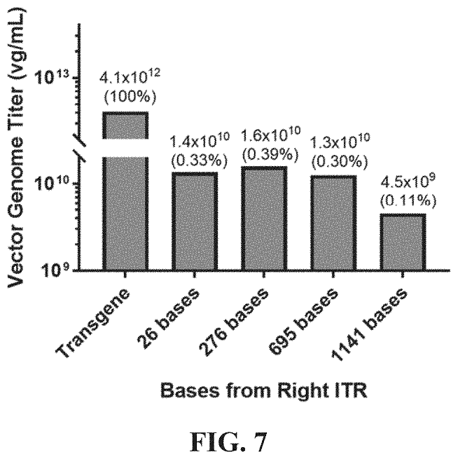

The High MW AADC and Low MW AADC were also analyzed by PCR and sequencing. Each was amplified using primers that hybridize at the internal edge of the ITR packaging elements. The PCR products were purified using a QIAGEN QIAquick PCR Cleanup Kit. The purified amplicons were analyzed by gel electrophoresis as shown in FIG. 6A, which showed that both High MW PCR and Low MW PCR resulted in the same size amplicon. The purified PCR products were also subjected to Sanger sequencing, and the sequencing reads for each material (High MW or Low MW) were assembled into separate sequences. All of the sequencing reads had homogenous nucleotide sequences, indicating that only a single population was sequenced, rather than a mixture. The consensus sequences were determined from at least four reads from the same region. The sequences were aligned and found to be the expected sequence and identical to each other. An illustration of the sequenced portion is shown in FIG. 6B. Titer measurements for amplified transgene sequence, and 26-, 276-, 695-, and 1141-base regions from the right ITR were used to measure contribution of baculovirus backbone to the packaged DNA. Results for these measurements are plotted in FIG. 7 and indicate that the approximately 1 kb of additional packaged DNA in the High MW AADC is not from backbone read-through. Together with the sequencing and endonuclease data, these results indicate that the approximately 1 kb of additional packaged DNA is a complementary portion of transgene sequence.

### Example 3

### Comparison of Expression Efficiencies

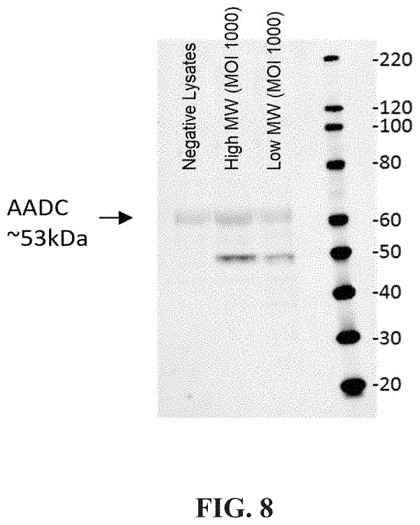

HT-1080 cells were transduced in vitro with either the High MW AADC or the Low MW AADC of Example 2. The cells were transduced at 1000 vector genomes per cell. The cells were incubated with vector for 34 hours. The cells were lysed, and the cell lysates were analyzed for AADC protein by Western blot. The AADC protein (approximately 53 kDa) was detected using a monoclonal antibody (Abeam ab211535) specific for the AADC enzyme. An illustrative blot is shown in FIG. 8. Both of the vectors produced AADC after addition to the HT-1080 cells, but expression from the High MW AADC was higher. An additional band was observed in both lysates at about 60 kDa, but this additional band was also observed in the negative control (untransduced cells), indicating the band was the result of non-specific binding of the antibody.

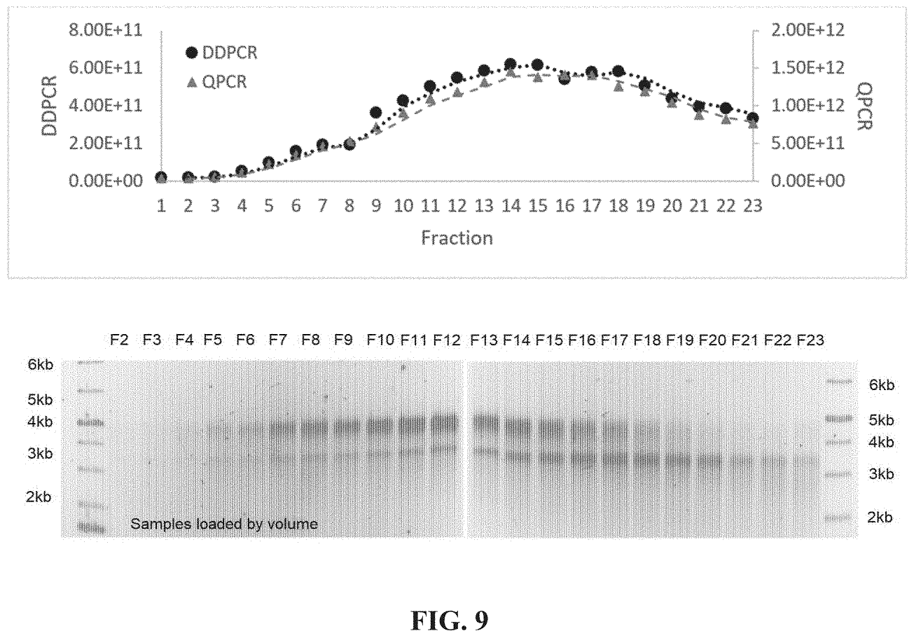

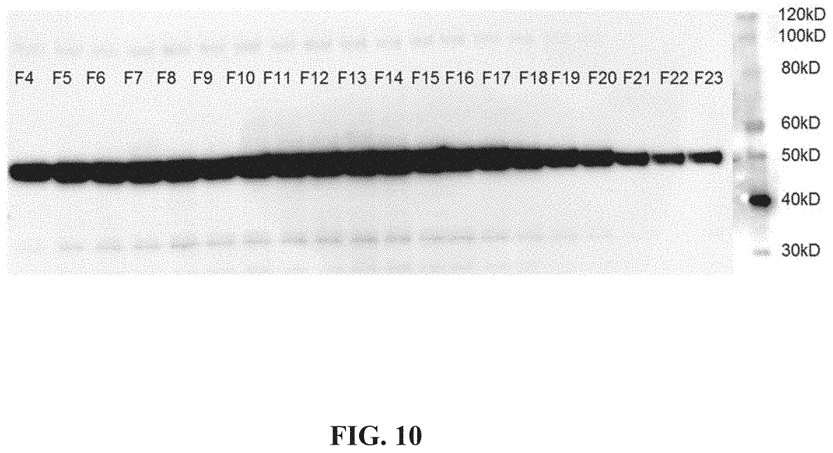

A separate 400 mL batch of rAAV2-hAADC, prepared as in Example 1, was separated using a shallow cesium chloride isopycnic gradient. The fractions from the gradient were analyzed by qPCR and ddPCR, results of which are illustrated in FIG. 9 (plot). Fractions F2-F23 were analyzed using an alkaline denaturing gel (FIG. 9, gel image). Fractions F7-F13 were enriched for the high molecular weight DNA form (approximately 4.5 kb), and fractions F19-F23 were enriched for the low molecular weight DNA form (approximately 3.5 kb). Each fraction was dialyzed against 1×PBS. The dialyzed fractions were then used to transduce HT-1080 cells at a multiplicity of infection (MOI) of 3×10³ vg/cell. After 48 hours, the cells were lysed, and lysates were analyzed by Western blot using the monoclonal AADC antibody. An illustrative image of the Western blot is shown in FIG. 10. Fractions enriched for the high molecular weight form produced a higher level of AADC expression than the fractions enriched for the low molecular weight form (compare, e.g., lanes F10 to F23 of FIG. 10),

Throughout this application various publications have been referenced. The disclosures of these publications in their entireties are hereby incorporated by reference in this application in order to more fully describe the state of the art to which this invention pertains. Although the invention has been described with reference to the examples provided above, it should be understood that various modifications can be made without departing from the spirit of the invention.

### VIII. EQUIVALENTS AND SCOPE

Those skilled in the art will recognize or be able to ascertain using no more than routine experimentation, many equivalents to the specific embodiments in accordance with the present disclosure described herein. The scope of the present disclosure is not intended to he limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the present disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known. to one of ordinary skill in the art, they may be excluded even if the exclusion is not set forth explicitly herein. Any particular embodiment of the compositions of the present disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the present disclosure in its broader aspects.

While the present disclosure has been described at some length and with some particularity with respect to the several described embodiments, it is not intended that it should he limited to any such particulars or embodiments or any particular embodiment, but it is to be construed with references to the appended claims so as to provide the broadest possible interpretation of such claims in view of the prior art and, therefore, to effectively encompass the intended scope of the present disclosure.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

We claim:

1. A partial self-complementary parvovirus genome comprising a payload construct, parvovirus inverted terminal repeats (ITRs) flanking the payload construct, and a self-complementary region flanking one of the ITRs, wherein the self-complementary region comprises a nucleotide sequence that is complementary to the payload construct and a length that is less the entire length of the payload construct.

2. The genome of claim 1, wherein the parvovirus is an adeno-associated virus (AAV).

3. The genome of claim 2, wherein the AAV is serotype AAV2.

4. The genome of any one of claims 1 to 3, wherein the payload construct encodes a protein of interest or produces a modulatory nucleic acid.

5. The genome of any one of claims 1 to 4, wherein the payload construct is 2.3 kilobases (kb), 2.4 kb, 2.5 kb, 2.6 kb, 2.7 kb, 2.8 kb, 2.9 kb, 3.0 kb, 3.1 kb, 3.2 kb, 3.3 kb, 3.4 kb, 3.5 kb or more in length.

6. The genome of any one of claims 1 to 5, wherein the self-complementary region is at least 50 bases, at least 100 bases, at least 200, at least 300 bases, at least 400 bases, at least 500 bases, at least 600 bases, at least 700 bases, at least 800 bases, at least 900 bases, at least 1,000 bases in length.

7. The genome of any one of claims 1 to 6, wherein the self-complementary region has a length of no more than 2.2 kb.

8. The genome of any one of claims 1 to 7, wherein the self-complementary region has a length between 50 bases and 2.0 kb.

9. The genome of any one of claims 1 to 8, wherein the genome has a total length of no more than 4.8 kb.

10. A parvovirus particle com the genome of any one of claims 1 to 9.

11. A population of parvovirus particles comprising a first sub-population of parvovirus particles each comprising the genome of any one of claims 1 to 9, wherein the first sub-population of parvovirus particles enriched with such parvovirus particles as compared to the population of parvovirus particles prior to its being enriched.

12. A population of parvovirus particles comprising a first sub-population of parvovirus particles and a second sub-population of parvovirus particles, wherein the first sub-population of parvovirus particles each comprise the genome of any one of claims 1 to 9, and wherein the second sub-population of parvovirus particles each comprise a genome that does not comprise the nucleotide sequence that is complementary to a portion of the payload construct.

13. The population of claim 12, the first sub-population of parvovirus particles is substantially isolated from the second sub-population of parvovirus particles.

14. The population of claim 12 or 13, wherein the first sub-population of parvovirus particles comprises a relative molar amount of at least 10%, at least 20%, at least 30%, at least 40%, at least 50%, at least 60%, at least 70%, at least 80%, at least 90%, at least 100%, at least 110%, at least 150%, at least 200%, at least 300%, at least 400%, at least 500%, at least 600%, at least 700%, at least 800%, at least 900%, at least 1,000% of the second sub-population of parvovirus particles.

15. A pharmaceutical composition comprising the parvovirus particle of claim 10 and a pharmaceutically acceptable carrier.

16. A pharmaceutical composition comprising the population of any one of claims 11 to 14 and a pharmaceutically acceptable carrier.

17. An insect cell comprising the genome of any one of claims 1 to 9.

18. The insect cell of claim 17, wherein the insect cell is a Spodoptera frugiperda pupal ovarian cell.

19. A plasmid vector encoding the genome of any one of claims 1 to 9.

20. A method of making a population of parvovirus particles comprising:
(a) culturing insect cells to produce a population of parvovirus particles; and
(b) harvesting the population of parvovirus particles produced by the insect cells,
wherein the harvested population of parvovirus particles include a first sub-population of parvovirus particles each having the partial self-complementary parvovirus genome of any one of claims 1 to 9.

21. A method of making a population of parvovirus particles comprising:
(a) culturing insect cells with a plasmid vector encoding the genome of any one of claims 1 to 9, to produce a population of partial self-complementary parvovirus genomes;
(b) culturing insect cells with the population of partial self-complementary parvovirus genomes to produce a population of parvovirus particles; and
(c) harvesting the population of parvovirus particles produced by the insect cells.

22. The method of claim 20 or claim 21, further comprising enriching the parvovirus particles for the first sub-population of parvovirus particles.

23. The method of claim 22, wherein the enriching step comprises density gradient centrifugation.

24. The method of claim 23, where the density gradient centrifugation is isopycnic centrifugation.

25. The method of any one of claims 20 to 24, wherein insect cells are Spodoptera frugiperda pupal ovarian cells.
